(19) United States
(12) Patent Application Publication
(10) Pub. No.: US 2020/0040390 A1
(43) Pub. Date: Feb. 6, 2020

(54) Methods for Sequencing Repetitive Genomic Regions

(71) Applicant: Centrillion Technologies, Inc., Palo Alto, CA (US)

(72) Inventors: John Chiang, Portland, OR (US); Wei ZHOU, Saratoga, CA (US)

(21) Appl. No.: 16/384,396

(22) Filed: Apr. 15, 2019

Related U.S. Application Data
(60) Provisional application No. 62/657,730, filed on Apr. 14, 2018.

(51) Int. Cl. C12Q 1/6869; C12Q 1/6827; G16B 30/00; G16B 50/30; G16B 50/20; G16B 45/00; G16B 40/10

(52) U.S. Cl. CPC C12Q 1/6869; C12Q 1/6827; G16B 30/00; C12Y 207/07007; G16B 50/20; G16B 45/00; G16B 40/10; G16B 50/30

(57) ABSTRACT

The present disclosure provides methods of sequencing a region of a nucleic acid and identifying mutations within the region. The disclosed methods may comprise constructing a nucleic acid fragments library of the region of the nucleic acid by using a deoxyribonuclease (DNase) to fragment amplification products of the region generated by long range polymerase chain reaction (LR-PCR) amplification. The sequencing method may also comprise a duplication analysis using an artificial sequence. The disclosed method may detect mutations within the region when the region comprises repetitive sequences.

### CROSS-REFERENCE

This application claims the benefit of U.S. Provisional Patent Application No. 62/657,730, filed Apr. 14, 2018, which application is entirely incorporated herein by reference.

### BACKGROUND

High-throughput sequencing has found application in many areas of modern biology from ecology and evolution, to gene discovery and discovery medicine. For example, in order to move forward the field of personalized medicine, the complete genotype and phenotype information of all geo-ethnic groups may need to be garnered. Having such information may permit physicians to tailor the treatment to each patient.

New sequencing methods, commonly referred to as Next Generation Sequencing (NGS) technologies, have promised to deliver fast, inexpensive and accurate genome information through sequencing. For example, high throughput NGS (HT-NGS) methods may allow scientists to obtain the desired sequence of genes with greater speed and at lower cost. Clinically screening a full genome for an individual's mutations may offer benefits both for pursuing personalized medicine and for uncovering genomic contributions to diseases.

Certain regions of the genome are highly complex and repetitive. These regions tend to be difficult to sequence using the short read technology such as the reversible terminator sequencing technology available from various vendors including Illumina. Various methods of sequencing library construction can be used to sequence the human genome. However, some of the library construction methods may be biased towards certain sequence features and may not capture certain complex genomic regions.

### SUMMARY

The present disclosure provides methods of sequencing a region of a nucleic acid and identifying mutations within the region. The disclosed methods may comprise constructing a nucleic acid fragments library of the region of the nucleic acid by using a deoxyribonuclease (DNase) to fragment amplification products of the region generated by long range polymerase chain reaction (LR-PCR) amplification. The sequencing method may also comprise a duplication analysis using an artificial sequence. The disclosed method may detect mutations within the region when the region comprises repetitive sequences.

An aspect of the present disclosure provides a method of constructing a sequencing library for a region of a target deoxyribonucleic acids (DNA), comprising: (a) performing a long range polymerase chain reaction (LR-PCR) amplification of the target DNA, thereby producing a plurality of amplified target DNA products; and (b) fragmenting the plurality of amplified target DNA products by using a deoxyribonuclease (DNase), thereby producing a plurality of fragments of the region of the target DNA; wherein the region of the target DNA comprises a plurality copies of a repetitive sequence.

In some embodiments of aspects provided herein, the region of the target DNA further comprises a plurality of variations selected from the group consisted of nucleotide variant, single base substitution, or small indel, transversion, translocation, inversion, deletion, truncation or gene truncation about 1, 2, 3, 4, 5, 6, 7, 8, 9, 10, 15 or 20 nucleotides in length, or a combination thereof. In some embodiments of aspects provided herein, the target DNA is RPGR-ORF15 region, mitochondria or STRC. In some embodiments of aspects provided herein, the LR-PCR amplification utilizes a plurality of primers, the primers are: (i) primers for RPGR-ORF15: Forward: AGCAGCCTGAGGCAATAGAA, Reverse: CAAAATTTACCAGTGCCTCCT; or (ii) primers for Mitochondria: Mitol (Mt1)—Forward: AAATCTTACCCCGCCTGTTT, Mitol (Mt1)—Reverse: AATTAGGCTGTGGGTGGTTG, and/or Mito2 (Mt2)—Forward: GCCATACTAGTCTTTGCCGC, Mito2 (Mt2)—Reverse: GCAGGTCAATTTCACTGGT; or (iii) primers for STRC: Forward: CAGCTCAGAGTTTTTGATAGGGCTTTCA, Reverse: AGGAAGCAGATCAAAGATTAGTGTCCCTT.

In some embodiments of aspects provided herein, a minimal depth coverage for the region of the target DNA is more than 900, 1,000, 2,000, 3,000, 4,000, 5,000, or 6,000 reads. In some embodiments of aspects provided herein, the. In some embodiments of aspects provided herein, the minimal depth coverage is about 10, 11, 12, 13, 14, 15, 16, 17, 18, 19, 20, or 21 times higher than another method, the another method using transposase-based Nextera fragmentation in (b). In some embodiments of aspects provided herein, the region of the target DNA is more than 1,000, 1,100, 1,200, 1,300, 1,400, 1,500, 1,600, 1,700, 1,800, 1,900, 2,000, 2,100, 2,200, 2,300, 2,400, or 2,500 bp in length. In some embodiments of aspects provided herein, the DNase is DNase I. In some embodiments of aspects provided herein, the. In some embodiments of aspects provided herein, the method further comprises, after (b), end repairing the plurality of fragments of the region of the target DNA, adding a single adenine to the 3′ ends of end repaired fragments using a template independent polymerase; and ligating an adaptor to each end of the repaired fragments comprising a 3′-adenine overhang.

Another aspect of the present disclosure provides a method of detecting at least one mutation within a region of a target deoxyribonucleic acids (DNA), comprising: (i) constructing the sequencing library for the region of the target DNA according to claim 1; (ii) sequencing the plurality of fragments of the region of the target DNA in the sequencing library by a next generation sequencing method, thereby acquiring a plurality of reads for the at least one mutation; and (iii) identifying the at least one mutation.

In some embodiments of aspects provided herein, the region of the target DNA further comprises a plurality of variations selected from the group consisted of nucleotide variant, single base substitution, or small indel, transversion, translocation, inversion, deletion, truncation or gene truncation about 1, 2, 3, 4, 5, 6, 7, 8, 9, 10, 15 or 20 nucleotides in length, or a combination thereof. In some embodiments of aspects provided herein, the target DNA is RPGR-ORF15 region, mitochondria or STRC. In some embodiments of aspects provided herein, a minimal depth coverage for the at least one mutation is more than 900, 1,000, 2,000, 3,000, 4,000, 5,000, or 6,000 reads. In some embodiments of aspects provided herein, the minimal depth coverage is about 10, 11, 12, 13, 14, 15, 16, 17, 18, 19, 20, or 21 times higher than another method, the another method using transposase-based Nextera fragmentation in (b) when constructing the sequencing library. In some embodiments of aspects provided herein, the. In some embodiments of aspects provided herein, the method further comprises, after (b) when constructing the sequencing library, end repairing the plurality of fragments of the region of the target DNA, adding a single adenine to the 3′ ends of end repaired fragments using a template independent polymerase; and ligating an adaptor to each end of the repaired fragments comprising a 3′-adenine overhang.

In some embodiments of aspects provided herein, the method further comprising, in (iii), conducting duplication analysis. In some embodiments of aspects provided herein, the duplication analysis detects a frameshift duplication or an in-frame duplication. In some embodiments of aspects provided herein, the duplication analysis comprises using an artificial reference sequence comprising contigs of about 140, 150, 160, 170, or 180 bp in length, wherein each of the contigs centers on a duplication breakpoint, and wherein two adjacent contigs are separated by a homopolymer “A” of about 40, 45, 50, 55, or 60 bp in length. In some embodiments of aspects provided herein, the duplication analysis detects a duplication mutation. In some embodiments of aspects provided herein, the duplication mutation is not detected by another method, the another method using transposase-based Nextera fragmentation in (b) when constructing the sequencing library.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an example distribution of read length of adapter-ligated fragments over the detected fragments when using Nextera as the fragmenting method of the amplicons.

FIG. 2 shows an example distribution of read length of adapter-ligated fragments over the detected fragments when using OneTube as the fragmenting method of the amplicons.

FIG. 3 depicts example mutation coverage curve and positions of missed mutations when Nextera is used the fragmenting method to generate the sequencing library.

FIG. 4 illustrates example mutation coverage curve of missed mutations, and example position and number of unique variants detected when OneTube is used the fragmenting method to generate the sequencing library.

FIG. 5 shows example alignment settings when analyzing sequencing results of the nucleic acid fragments.

FIG. 6 depicts duplication analysis when using an example artificial reference sequence to detect a duplication mutation.

FIG. 7 illustrates duplication zygosity testing of a mixed sample containing a negative control and a sample homozygous for the region of the target nucleic acid.

### DETAILED DESCRIPTION

The second generation sequencing (NGS) approaches, involving sequencing by synthesis (SBS) have experienced a rapid development as data produced by these new technologies mushroomed exponentially. The SBS approach may have shown promise as a new sequencing platform. Despite remarkable progress in last two decades, there remains much room for the development for a clinical relevant NGS approach to perform high-throughput, accurate, and clinically relevant analysis of patient samples.

For example, mutations in the ORF15 region of RPGR may account for roughly half of all X-linked retinitis pigmentosa (RP) cases, providing a key target for recently launched human RPGR gene therapy trials. Despite its significance, a robust and reliable high throughput method for the detection of ORF15 mutations has yet to be validated. Here, after much refinement, the inventors developed the first clinically validated next-generation sequencing (NGS) method, complete with test accuracy and coverage data, for the detection of mutations in this difficult-to-sequence region of genetic information.

Retinitis pigmentosa (RP, OMIM #268,000) may be the most commonly diagnosed inherited retinal dystrophy (IRD). It may be clinically and genetically heterogeneous, with at least 64 causative genes currently identified. The more severe, X-linked form of RP (xlRP) may constitute 10-20% of all RP cases. Roughly 9% of families may have an autosomal dominant form of RP (adRP) and 15% of male sporadic cases can be attributed to mutations in the X-linked genes, Retinitis pigmentosa 2 (RP2; MIM 300757) and Retinitis pigmentosa GTPase regulator (RPGR; MIM 312610). RPGR mutations account for >70% of these cases and as such, may be the most common RP gene.

RPGR may encode several isoforms, but only the largest of these, Isoform C (NM_001034853), can be highly expressed in the retina and involved in the pathogenesis of RP. This isoform, also known as RPGR ORF15, spans 4767 nucleotides encoding a 1152-amino acid protein (NP_001030025). Over 60% of all RPGR mutations can be clustered to its unique terminal exon, ORF15 (c.1754-3459) that may encode a 567-amino acid C-terminus rich in glutamic acid and glycine. One reason for this may be the slippage of DNA polymerase on the highly repetitive, 1 kb, purine-rich region (c.2184-3162).

Therefore, there is a need for accurate detection of ORF15 mutations which can be central to the diagnosis of this condition and subsequent genetic counseling and family planning decisions. Looking forward, a robust, accurate and scalable test for ORF15 can be necessary for personalized medicine strategies such as participation in gene-therapy clinical trials and the prescription of approved treatments that may arise from these.

Despite this impending necessity, current clinical testing of ORF15 still relies on traditional Sanger sequencing, long after Next Generation Sequencing (NGS) has become the clinical standard for the genetic testing of IRDs. This can be attributed to the highly repetitive, difficult-to-sequence, region of ORF15 that amplifies existing limitations of NGS methods. Herein disclosed is a blind validation of a new NGS method for ORF15. Specificity and sensitivity of this new NGS method are presented, thus documenting the first clinically validated sequencing method of one of the most difficult-to-sequence regions in the genome.

RP may be a predominant form of inherited retinal disease, with a reported prevalence of around 1 in 4000. X-linked gene, RPGR, is the most common causative gene of all RP disease genes currently identified. This is due to a highly repetitive and thus unstable 1 kb sequence of tandem repeats within ORF15 of Isoform C, which constitutes a mutational hotspot. Repetitive sequences of tandem repeats may be a common cause of heritable disease. Mutation of the highly repetitive and unstable ORF15 region of RPGR may cause 25% to 70% of xlRP cases. However, different from other repeat expansion diseases, mutations in ORF15 can be mostly frameshift mutations caused by small deletions or insertions.

Therefore, accurate mutation detection in this region can be critical to the diagnosis and management of this condition, while a fast-turn-around time may also be an ever-increasing expectation. However, for ORF15, satisfying these requirements may be difficult. As for other similarly repetitive regions, ORF15 can be refractory to variant detection using traditional NGS methods including the Nextera NGS method. The Sanger sequencing of ORF15 can be labor-intensive, time-consuming, and subject to allele dropout. Coupled with increasing clinical volumes and the demand for a more timely turnaround of test samples, there is an urgent need for an accurate, high-throughput mutation detection method to assist in the diagnosis and management of xlRP.

Facing these problems, there is a need to develop a new NGS sequencing method with better accuracy and speed. The present disclosure presents a clinically validated NGS method for ORF15 screening. For the first time, a complete analysis of ORF15 using NGS method in a standardized clinical pipeline was accomplished. Through a blind test of 145 Sanger-sequenced samples, followed by further validation using an additional 81 Sanger-sequenced clinical samples, the present disclosure can present a highly accurate and sensitive method for detection of ORF15 mutations in a clinical setting.

Sequencing-by-Synthesis (SBS) and Single-Base-Extension (SBE) Sequencing

Several techniques are available to achieve high-throughput sequencing. (See, Ansorge; Metzker; and Pareek et al., “Sequencing technologies and genome sequencing,” J. Appl. Genet., 52(4):413-435, 2011, and references cited therein). The SBS method is a commonly employed approach, coupled with improvements in polymerase chain reaction (PCR), such as emulsion PCR (emPCR), to rapidly and efficiently determine the sequence of many fragments of a nucleotide sequence in a short amount of time. In SBS, nucleotides are incorporated by a polymerase enzyme and because the nucleotides are differently labeled, the signal of the incorporated nucleotide, and therefore the identity of the nucleotide being incorporated into the growing synthetic polynucleotide strand, are determined by sensitive instruments, such as cameras.

SBS methods commonly employ reversible terminator nucleic acids, i.e. bases which contain a covalent modification precluding further synthesis steps by the polymerase enzyme once incorporated into the growing stand. This covalent modification can then be removed later, for instance using chemicals or specific enzymes, to allow the next complementary nucleotide to be added by the polymerase. Other methods employ sequencing-by-ligation techniques, such as the Applied Biosystems SOLiD platform technology. Other companies, such as Helicos, provide technologies that are able to detect single molecule synthesis in SBS procedures without prior sample amplification, through use of very sensitive detection technologies and special labels that emit sufficient light for detection. Pyrosequencing is another technology employed by some commercially available NGS instruments. The Roche Applied Science 454 GenomeSequencer, involves detection of pyrophosphate (pyrosequencing). (See, Nyren et al., “Enzymatic method for continuous monitoring of inorganic pyrophosphate synthesis,” Anal. Biochem., 151:504-509, 1985; see also, US Patent Application Publication Nos. 2005/0130173 and 2006/0134633; U.S. Pat. Nos. 4,971,903, 6,258,568 and 6,210,891).

Sequencing using the presently disclosed reversible terminator molecules may be performed by any means available. Generally, the categories of available technologies include, but are not limited to, sequencing-by-synthesis (SBS), sequencing by single-base-extension (SBE), sequencing-by-ligation, single molecule sequencing, and pyrosequencing, etc. The method most applicable to the present compounds, compositions, methods and kits is SBS. Many commercially available instruments employ SBS for determining the sequence of a target polynucleotide. Some of these are briefly summarized below.

One method, used by the Roche Applied Science 454 GenomeSequencer, involves detection of pyrophosphate (pyrosequencing). (See, Nyren et al., “Enzymatic method for continuous monitoring of inorganic pyrophosphate synthesis,” Anal. Biochem., 151:504-509, 1985). As with most methods, the process begins by generating nucleotide fragments of a manageable length that work in the system employed, i.e. about 400-500 bp. (See, Metzker, Michael A., “Sequencing technologies—the next generation,” Nature Rev. Gen., 11:31-46, 2010). Nucleotide primers are ligated to either end of the fragments and the sequences individually amplified by binding to a bead followed by emulsion PCR. The amplified DNA is then denatured and each bead is then placed at the top end of an etched fiber in an optical fiber chip made of glass fiber bundles. The fiber bundles have at the opposite end a sensitive charged-couple device (CCD) camera to detect light emitted from the other end of the fiber holding the bead. Each unique bead is located at the end of a fiber, where the fiber itself is anchored to a spatially-addressable chip, with each chip containing hundreds of thousands of such fibers with beads attached. Next, using an SBS technique, the beads are provided a primer complementary to the primer ligated to the opposite end of the DNA, polymerase enzyme and only one native nucleotide, i.e., C, or T, or A, or G, and the reaction allowed to proceed. Incorporation of the next base by the polymerase releases light which is detected by the CCD camera at the opposite end of the bead. (See, Ansorge, Wilhelm J., “Next-generation DNA sequencing techniques,” New Biotech., 25(4):195-203, 2009). The light is generated by use of an ATP sulfurylase enzyme, inclusion of adenosine 5′ phosphosulferate, luciferase enzyme and pyrophosphate. (See, Ronaghi, M., “Pyrosequencing sheds light on DNA sequencing,” Genome Res., 11(1):3-11, 2001).

Long Range Polymerase Chain Reaction (LR-PCR)

Polymerase chain reaction (PCR) has been described in, for example, U.S. Pat. Nos. 4,683,195, 4,683,202, and 4,800,159; K. Mullis, Cold Spring Harbor Symp. Quant. Biol., 51:263-273 (1986); and C. R. Newton & A. Graham, Introduction to Biotechniques: PCR, 2.sup.nd Ed., Springer-Verlag (New York: 1997), the disclosures of which are incorporated entirely herein by reference. In some cases, the methods disclosed herein describe processes to amplify a nucleic acid sample target using PCR amplification extension primers which hybridize with the sample target. As the PCR amplification primers are extended, using a DNA polymerase (for example, a thermostable DNA polymerase), more sample target can be made so that more primers can be used to repeat the process, thus amplifying the sample target sequence. In some cases, the reaction conditions can be cycled between those conducive to hybridization and nucleic acid polymerization, and those that result in the denaturation of duplex molecules.

Example methods for performing long range PCR may be found, for example, in U.S. Pat. No. 5,436,149; Barnes, Proc. Natl. Acad. Sci. USA 91:2216-2220 (1994); Tellier et al., Methods in Molecular Biology, Vol. 226, PCR Protocols, 2nd Edition, pp. 173-177; and, Cheng et al., Proc. Natl. Acad. Sci. 91:5695-5699 (1994); the contents of which are incorporated entirely herein by reference. In some cases, long range PCR may involve one DNA polymerase. In some cases, long range PCR may involve more than one DNA polymerase. When using a combination of polymerases in long range PCR, the methods may include one polymerase having 3′→5′ exonuclease activity, which may provide high fidelity generation of the PCR product from the DNA template. In some cases, a non-proofreading polymerase, which may be the main polymerase, may also be used in conjunction with the proofreading polymerase in long range PCR reactions. Long range PCR can also be performed using commercially available kits, such as LA PCR kit available from Takara Bio Inc. Polymerase enzymes having 3′→5′ exonuclease proofreading activity may include TaKaRa LA Taq (Takara Shuzo Co., Ltd.) and Pfu (Stratagene), Vent, Deep Vent (New England Biolabs).

A commercially available instrument, called the Genome Analyzer, also utilizes SBS technology. (See, Ansorge, at page 197). Similar to the Roche instrument, sample DNA is first fragmented to a manageable length and amplified. The amplification step is somewhat unique because it involves formation of about 1,000 copies of single-stranded DNA fragments, called polonies. Briefly, adapters are ligated to both ends of the DNA fragments, and the fragments are then hybridized to a surface having covalently attached thereto primers complimentary to the adapters, forming tiny bridges on the surface. Thus, amplification of these hybridized fragments yields small colonies or clusters of amplified fragments spatially co-localized to one area of the surface. SBS is initiated by supplying the surface with polymerase enzyme and reversible terminator nucleotides, each of which is fluorescently labeled with a different dye. Upon incorporation into the new growing strand by the polymerase, the fluorescent signal is detected using a CCD camera. The terminator moiety, covalently attached to the 3′ end of the reversible terminator nucleotides, is then removed as well as the fluorescent dye, providing the polymerase enzyme with a clean slate for the next round of synthesis. (Id., see also, U.S. Pat. No. 8,399,188; Metzker, at pages 34-36).

Polymerase Enzymes Used in SBS/SBE Sequencing

As already commented upon, one of the key challenges facing SBS or SBE technology is finding reversible terminator molecules capable of being incorporated by polymerase enzymes efficiently and which provide a blocking group that can be removed readily after incorporation. Thus, to achieve the presently claimed methods, polymerase enzymes must be selected which are tolerant of modifications at the 3′ and 5′ ends of the sugar moiety of the nucleoside analog molecule. Such tolerant polymerases are known and commercially available.

BB Preferred polymerases lack 3′-exonuclease or other editing activities. As reported elsewhere, mutant forms of 9° N-7(exo-) DNA polymerase can further improve tolerance for such modifications (WO 2005024010; WO 2006120433), while maintaining high activity and specificity. An example of a suitable polymerase is THERMINATOR™ DNA polymerase (New England Biolabs, Inc., Ipswich, Mass.), a Family B DNA polymerase, derived from Thermococcus species 9° N-7. The 9° N-7(exo-) DNA polymerase contains the D141A and E143A variants causing 3’-5′ exonuclease deficiency. (See, Southworth et al., “Cloning of thermostable DNA polymerase from hyperthermophilic marine Archaea with emphasis on Thermococcus species 9° N-7 and mutations affecting 3′-5′ exonuclease activity,” Proc. Natl. Acad. Sci. USA, 93(11): 5281-5285, 1996). THERMINATOR™ I DNA polymerase is 9° N-7(exo-) that also contains the A485L variant. (See, Gardner et al., “Acyclic and dideoxy terminator preferences denote divergent sugar recognition by archaeon and Taq DNA polymerases,” Nucl. Acids Res., 30:605-613, 2002). THERMINATOR™ III DNA polymerase is a 9° N-7(exo-) enzyme that also holds the L4085, Y409A and P410V mutations. These latter variants exhibit improved tolerance for nucleotides that are modified on the base and 3′ position. Another polymerase enzyme useful in the present methods and kits is the exo-mutant of KOD DNA polymerase, a recombinant form of Thermococcus kodakaraensis KOD1 DNA polymerase. (See, Nishioka et al., “Long and accurate PCR with a mixture of KOD DNA polymerase and its exonuclease deficient mutant enzyme,” J. Biotech., 88:141-149, 2001). The thermostable KOD polymerase is capable of amplifying target DNA up to 6 k bp with high accuracy and yield. (See, Takagi et al., “Characterization of DNA polymerase from Pyrococcus sp. strain KOD1 and its application to PCR,” App. Env. Microbiol., 63(11):4504-4510, 1997). Others are Vent (exo-), Tth Polymerase (exo-), and Pyrophage (exo-) (available from Lucigen Corp., Middletown, Wis., US). Another non-limiting exemplary DNA polymerase is the enhanced DNA polymerase, or EDP. (See, WO 2005/024010).

When sequencing using SBE, suitable DNA polymerases include, but are not limited to, the Klenow fragment of DNA polymerase I, SEQUENASE™ 1.0 and SEQUENASE™ 2.0 (U.S. Biochemical), T5 DNA polymerase, Phi29 DNA polymerase, THERMOSEQUENASE™ (Taq polymerase with the Tabor-Richardson mutation, see Tabor et al., Proc. Natl. Acad. Sci. USA, 92:6339-6343, 1995) and others known in the art or described herein. Modified versions of these polymerases that have improved ability to incorporate a nucleotide analog of the disclosure can also be used.

Further, it has been reported that altering the reaction conditions of polymerase enzymes can impact their promiscuity, allowing incorporation of modified bases and reversible terminator molecules. For instance, it has been reported that addition of specific metal ions, e.g., Mn²⁺, to polymerase reaction buffers yield improved tolerance for modified nucleotides, although at some cost to specificity (error rate). Additional alterations in reactions may include conducting the reactions at higher or lower temperature, higher or lower pH, higher or lower ionic strength, inclusion of co-solvents or polymers in the reaction, and the like.

Random or directed mutagenesis may also be used to generate libraries of mutant polymerases derived from native species; and the libraries can be screened to select mutants with optimal characteristics, such as improved efficiency, specificity and stability, pH and temperature optimums, etc. Polymerases useful in sequencing methods are typically polymerase enzymes derived from natural sources. Polymerase enzymes can be modified to alter their specificity for modified nucleotides as described, for example, in WO 01/23411, U.S. Pat. No. 5,939,292, and WO 05/024010. Furthermore, polymerases need not be derived from biological systems.

The terminology used herein is for the purpose of describing particular cases only and is not intended to be limiting. As used herein, the singular forms “a”, “an” and “the” can be intended to include the plural forms as well, unless the context clearly indicates otherwise. Furthermore, to the extent that the terms “including”, “includes”, “having”, “has”, “with”, or variants thereof can be used in either the detailed description and/or the claims, such terms can be intended to be inclusive in a manner similar to the term “comprising”.

The term “about” or “approximately” can mean within an acceptable error range for the particular value as determined by one of ordinary skill in the art, which may depend in part on how the value is measured or determined, i.e., the limitations of the measurement system. For example, the term “about” as used herein indicates the value of a given quantity varies by +/−10% of the value, or optionally +/−5% of the value, or in some embodiments, by +/−1% of the value so described. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values may be described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. For example, an active agent that is “substantially localized” in an organ can indicate that about 90% by weight of an active agent, salt, or metabolite can be present in an organ relative to a total amount of an active agent, salt, or metabolite. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “fragment” as used herein generally refers to a fraction of the original DNA sequence or RNA sequence of the particular region.

As used herein, nucleotides are abbreviated with 3 letters. The first letter indicates the identity of the nitrogenous base (e.g. A for adenine, G for guanine), the second letter indicates the number of phosphates (mono, di, tri), and the third letter is P, standing for phosphate. Nucleoside triphosphates that contain ribose as the sugar, ribonucleoside triphosphates, are conventionally abbreviated as NTPs, while nucleoside triphosphates containing deoxyribose as the sugar, deoxyribonucleoside triphosphates, are abbreviated as dNTPs. For example, dATP stands for deoxyribose adenine triphosphate. NTPs are the building blocks of RNA, and dNTPs are the building blocks of DNA.

The term “target nucleic acid” as used herein generally refers to the nucleic acid fragment targeted for detection using hybridization assays of the present disclosure. Sources of target nucleic acids may be isolated from organisms, including mammals, or pathogens to be identified, including viruses and bacteria. Additionally target nucleic acids may also be from synthetic sources. Target nucleic acids may be or may not be amplified via standard replication/amplification procedures to produce nucleic acid sequences.

The term “nucleic acid sequence” or “nucleotide sequence” as used herein generally refers to nucleic acid molecules with a given sequence of nucleotides, of which it may be desired to know the presence or amount. The nucleotide sequence can comprise ribonucleic acid (RNA) or DNA, or a sequence derived from RNA or DNA. Examples of nucleotide sequences are sequences corresponding to natural or synthetic RNA or DNA including genomic DNA and messenger RNA. The length of the sequence can be any length that can be amplified into nucleic acid amplification products, or amplicons, for example up to about 20, 50, 100, 200, 300, 400, 500, 600, 700, 800, 1,000, 1,200, 1,500, 2,000, 5,000, 10,000 or more than 10,000 nucleotides in length.

The term “template” as used herein generally refers to individual polynucleotide molecules from which another nucleic acid, including a complementary nucleic acid strand, may be synthesized by a nucleic acid polymerase. In addition, the template may be one or both strands of the polynucleotides that are capable of acting as templates for template-dependent nucleic acid polymerization catalyzed by the nucleic acid polymerase. Use of this term may not be taken as limiting the scope of the present disclosure to polynucleotides which are actually used as templates in a subsequent enzyme-catalyzed polymerization reaction.

The term “repetitive genomic sequences” or “repetitive sequences” or “repeat sequences” or “repetitive elements” as used herein generally refer to long sequence stretches that occur two or more times in the genome with high similarity between occurrences. For example, a repetitive sequence may appear multiple times in a region of the DNA, separated by the different DNA sequences. For example, repetitive sequences may be categorized in sequence families and may be broadly classified as interspersed repetitive DNA (see, e.g., Jelinek and Schmid, Ann. Rev. Biochem. 51:831-844, 1982; Hardman, Biochem J. 234:1-11, 1986; and Vogt, Hum. Genet. 84:301-306, 1990) or tandemly repeated DNA. Repetitive sequences may include satellite, minisatellite, and microsatellite DNA. In humans, interspersed repetitive DNA may include, but are not limited to, Alu sequences, short interspersed nuclear elements (SINE) and long interspersed nuclear elements (LINEs), endogenous retroviruses (ERVs), and certain transposons such as L and P element sequences. The categorization of repetitive elements and families of repetitive elements and their reference consensus sequences may be found in public databases (e.g., repbase (version 18.10)—Genetic Information Research Institute (Jurka et al., Cytogenet Genome Res 2005; 110:462-7)). In some cases, a repetitive sequence may be a segment of DNA that contains a sequence of nucleotides that is repeated for at least 3, 5, 10, 15, 20, 30, 40, 50, 60, 80, or 100 or more times. Repetitive sequences can include single nucleotide repeats (homopolymer stretches, e.g., poly A or poly T tails), di-nucleotide repeats (e.g., ATAT or AGAG), tri-nucleotide repeats, tetranucleotide repeats, telomeric repetitive elements and the like. ALU elements are a type of SINE element, roughly 300 base pairs in length.

The term “PCR” or “Polymerase chain reaction” as used herein generally refers to the enzymatic replication of nucleic acids, which uses thermal cycling for example to denature, extend and anneal the nucleic acids.

The terms a “forward primer” and a “reverse primer as used herein generally refer to a pair of primers that can bind to a template nucleic acid, and under proper amplification conditions produce an amplification product. If the forward primer is binding to the sense strand then the reverse primer is binding to antisense strand. Alternatively, if the forward primer is binding to the antisense strand then the reverse primer is binding to sense strand. The forward or reverse primer can bind to either strand as long as the other reverse or forward primer binds to the opposite strand.

A “forward primer” and a “reverse primer” constitute a pair of primers that can bind to a template nucleic acid and under proper amplification conditions produce an amplification product. If the forward primer is binding to the sense strand then the reverse primer is binding to antisense strand. Alternatively, if the forward primer is binding to the antisense strand then the reverse primer is binding to sense strand. In essence, the forward or reverse primer can bind to either strand as long as the other reverse or forward primer binds to the opposite strand

The term “label” or “detectable label” as used herein generally refers to any moiety or property that is detectable, or allows the detection of an entity which is associated with the label. For example, a nucleotide, oligo- or polynucleotide that comprises a fluorescent label may be detectable. In some cases, a labeled oligo- or polynucleotide permits the detection of a hybridization complex, for example, after a labeled nucleotide has been incorporated by enzymatic means into the hybridization complex of a primer and a template nucleic acid. A label may be attached covalently or non-covalently to a nucleotide, oligo- or polynucleotide. In some cases, a label can, alternatively or in combination: (i) provide a detectable signal; (ii) interact with a second label to modify the detectable signal provided by the second label, e.g., FRET; (iii) stabilize hybridization, e.g., duplex formation; (iv) confer a capture function, e.g., hydrophobic affinity, antibody/antigen, ionic complexation, or (v) change a physical property, such as electrophoretic mobility, hydrophobicity, hydrophilicity, solubility, or chromatographic behavior. Labels may vary widely in their structures and their mechanisms of action. Examples of labels may include, but are not limited to, fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like. Fluorescent labels may include dyes of the fluorescein family, dyes of the rhodamine family, dyes of the cyanine family, or a coumarine, an oxazine, a boradiazaindacene or any derivative thereof. Dyes of the fluorescein family include, e.g., FAM, HEX, TET, JOE, NAN and ZOE. Dyes of the rhodamine family include, e.g., Texas Red, ROX, R110, R6G, and TAMRA. FAM, HEX, TET, JOE, NAN, ZOE, ROX, R110, R6G, and TAMRA are commercially available from, e.g., Perkin-Elmer, Inc. (Wellesley, Mass., USA), Texas Red is commercially available from, e.g., Thermo Fisher Scientific, Inc. (Grand Island, N.Y., USA). Dyes of the cyanine family include, e.g., CY2, CY3, CY5, CY5.5 and CY7, and are commercially available from, e.g., GE Healthcare Life Sciences (Piscataway, N.J., USA).

The term “DNA polymerase” as used herein generally refers to a cellular or viral enzyme that synthesizes DNA molecules from their nucleotide building blocks.

As used herein, the solid substrate used can be biological, non-biological, organic, inorganic, or a combination of any of these. The substrate can exist as one or more particles, strands, precipitates, gels, sheets, tubing, spheres, containers, capillaries, pads, slices, films, plates, slides, or semiconductor integrated chips, for example. The solid substrate can be flat or can take on alternative surface configurations. For example, the solid substrate can contain raised or depressed regions on which synthesis or deposition takes place. In some examples, the solid substrate can be chosen to provide appropriate light-absorbing characteristics. For example, the substrate can be a polymerized Langmuir Blodgett film, functionalized glass (e.g., controlled pore glass), silica, titanium oxide, aluminum oxide, indium tin oxide (ITO), Si, Ge, GaAs, GaP, SiO₂, SiN₄, modified silicon, the top dielectric layer of a semiconductor integrated circuit (IC) chip, or any one of a variety of gels or polymers such as (poly)tetrafluoroethylene, (poly)vinylidenedifluoride, polystyrene, polycarbonate, polydimethylsiloxane (PDMS), polymethylmethacrylate (PMMA), polycyclicolefins, or combinations thereof.

Solid substrates can comprise polymer coatings or gels, such as a polyacrylamide gel or a PDMS gel. Gels and coatings can additionally comprise components to modify their physicochemical properties, for example, hydrophobicity. For example, a polyacrylamide gel or coating can comprise modified acrylamide monomers in its polymer structure such as ethoxylated acrylamide monomers, phosphorylcholine acrylamide monomers, betaine acrylamide monomers, and combinations thereof.

The term “complementary” as used herein generally refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A “polynucleotide sequence” or “nucleotide sequence” as used herein generally refers to a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

Two polynucleotides “hybridize” when they associate to form a stable duplex, e.g., under relevant assay conditions. Nucleic acids hybridize due to a variety of well characterized physicochemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York), as well as in Ausubel, infra.

The term “polynucleotide” (and the equivalent term “nucleic acid”) encompasses any physical string of monomer units that can be corresponded to a string of nucleotides, including a polymer of nucleotides, e.g., a typical DNA or RNA polymer, peptide nucleic acids (PNAs), modified oligonucleotides, e.g., oligonucleotides comprising nucleotides that are not typical to biological RNA or DNA, such as 2′-O-methylated oligonucleotides, and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

The term “oligonucleotide” as used herein generally refers to a nucleotide chain. In some cases, an oligonucleotide is less than 200 residues long, e.g., between 15 and 100 nucleotides long. The oligonucleotide can comprise at least or about 1, 2, 3, 4, 5, 6, 7, 8, 9, 10, 15, 20, 25, 30, 35, 40, 45, or 50 bases. The oligonucleotides can be from about 3 to about 5 bases, from about 1 to about 50 bases, from about 8 to about 12 bases, from about 15 to about 25 bases, from about 25 to about 35 bases, from about 35 to about 45 bases, or from about 45 to about 55 bases. The oligonucleotide (also referred to as “oligo”) can be any type of oligonucleotide (e.g., a primer). Oligonucleotides can comprise natural nucleotides, non-natural nucleotides, or combinations thereof.

Targets for Assays

Genetic materials useful as targets for the present disclosure may include, but are not limited to, DNA and RNA. There may be many different types of RNA and DNA, all of which have been and continue to be the subject of great study and experimentation. Targets of DNA may include, but are not limited to, genomic DNA (gDNA), chromosomal DNA, mitochondrial DNA (mtDNA), plasmid DNA, ancient DNA (aDNA), all forms of DNA including A-DNA, B-DNA, and Z-DNA, branched DNA, and non-coding DNA. Forms of RNA that may be sequenced using the present methods and compositions include, but are not limited to, messenger RNA (mRNA), ribosomal RNA (rRNA), microRNA, small RNA, snRNA and non-coding RNA. (See, Limbach et al., “Summary: The modified nucleosides of RNA,” Nuc. Acids Res., 22(12):2183-2196, 1994).

Nucleotides may include, but are not limited to, the naturally occurring nucleotides G, C, A, T and U, as well as rare forms, such as, Inosine, Xanthosine, 7-methylguanosine, dihydrouridine, 5-methylcytosine, and pseudouridine, including methylated forms of G, A, T, and C, and the like. (See, for instance, Korlach et al., “Going beyond five bases in DNA sequencing,” Curr. Op. Struct. Biol., 22(3):251-261, 2012; and U.S. Pat. No. 5,646,269). Nucleosides may also be non-naturally occurring molecules, such as those comprising 7-deazapurine, pyrazolo[3,4-d]pyrimidine, propynyl-dN, or other analogs or derivatives. Example nucleosides include ribonucleosides, deoxyribonucleosides, dideoxyribonucleosides, carbocyclic nucleosides, and the like.

Samples

Generally, any sample containing genetic material possessing a sequence of nucleotides of interest may be amenable to the present disclosure. Samples may be obtained from eukaryotes, prokaryotes and archaea. For example, samples containing genetic material whose sequence may be determined using the present disclosure include those obtained from, for instance, bacteria, bacteriophage, virus, transposons, mammals, plants, fish, insects, etc.

Samples may be human in origin and may be obtained from any human tissue containing genetic material. Generally, the samples may be fluid samples, such as, but not limited to normal and pathologic bodily fluids and aspirates of those fluids.

Purification/Isolation of DNA Sample for Assays

To prepare a sample for determination or detection of the sequence of genetic information contained therein, one may isolate and/or purify the genetic material away from other components in the original sample. There may be methods for purifying nucleic acid material from a sample. (See, for instance, Kennedy, S., “Isolation of DNA and RNA from soil using two different methods optimized with Inhibitor Removal Technology® (IRT),”BioTechniques, p. 19, November 2009; Molecular Cloning—A Laboratory Manual (Fourth Edition) Green, M., and Sambrook, J., Cold Spring Harbor Laboratory Press, US, 2012; Methods and Tools in Biosciences and Medicine, Techniques in molecular systematics and evolution, DeSalle et al. Ed., 2002, Birkhauser Verlag Basel/Switzerland; Keb-Llanes et al., Plant Molecular Biology Reporter, 20:299a-299e, 2002).

Fragmentation of DNA Sample to Produce Targets for Assays

Fragmentation of the polynucleotide targets in a DNA sample may be conducted prior to utilization of the various methods and devices disclosed in the present disclosure. These methods may include sonication, nebulization, hydro-shearing and shearing by other mechanical methods, such as, by using beads, needle shearing, French pressure cells, and acoustic shearing, etc., restriction digest, and other enzymatic methods such as use of various combinations of nucleases (DNase, exonucleases, endonucleases, etc.), as well as transposon-based methods. (See, Knierim et al., “Systematic Comparison of Three Methods for Fragmentation of Long-Range PCR Products for Next Generation Sequencing,” PLoS One, 6(11): e28240, 2011; Quail, M. A., “DNA: Mechanical Breakage,” Nov. 15, 2010, eLS; Sambrook, J., “Fragmentation of DNA by Nebulization,” Cold Spring Harb. Protoc., doi:10.1101/pdb.prot4539, 2006). Generally, the goal can be to obtain polynucleotides of a base pair (bp) size range that is amenable to the assay method chosen. For instance, the fragments may be about 50 bp, about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp or more.

In one embodiment, the fragmentation of the DNA sample may be performed by chemical, enzymatic, or physical methods. The fragmenting may be performed by enzymatic or mechanical methods. The mechanical methods may be sonication or physical shearing. The enzymatic methods may be performed by digestion with nucleases (e.g., Deoxyribonuclease I (DNase I)) or one or more restriction endonucleases. In some embodiments, the fragmentation results in ends for which the sequence may not be known.

In another embodiment, the enzymatic methods may be using DNase I. DNase I can be an enzyme that nonspecifically cleaves double-stranded DNA (dsDNA) to release 5′-phosphorylated di-, tri-, and oligonucleotide products. DNase I may have activity in buffers containing Mn²⁺, Mg²⁺ and Ca²⁺. The purpose of the DNase I digestion step can be to fragment a large DNA genome into smaller fragments of a library. The cleavage characteristics of DNase I may result in random digestion of the substrate DNA (i.e., no sequence bias for breaking the DNA molecule) and may result in the predominance of blunt-ended dsDNA fragments when used in the presence of manganese-based buffers (Melgar and Goldthwait, “Deoxyribonucleic acid nucleases. II. The effects of metal on the mechanism of action of deoxyribonuclease I,” J. Biol. Chem. 243(17):4409-16, 1968). The range of digestion products generated following DNase I treatment of genomic templates may depend on three factors: i) amount of enzyme used (units); ii) temperature of digestion (° C.); and iii) incubation time (minutes). The DNase I digestion may be optimized to yield genomic libraries with a size range from about 50 to about 700 bp.

In one embodiment, the DNase I may digest a large substrate DNA or whole genome DNA for about 1 or about 2 minutes to generate a population of fragmented polynucleotides. In another embodiment, the DNase I digestion may be performed at a temperature between about 10° C. to about 37° C. In yet another embodiment, the digested DNA fragments may be between 50 bp to 700 bp in length.

Furthermore, in some embodiments, the digestion of genomic DNA (gDNA) substrates with DNase I in the presence of Mn²⁺ may yield fragments of DNA that are either blunt-ended or have protruding termini with one or two nucleotides in length. In one embodiment, an increased number of blunt ends may be created with Pfu DNA polymerase. Use of Pfu DNA polymerase for fragment polishing may result in the fill-in of 5′ overhangs. Additionally, Pfu DNA polymerase may result in the removal of single and double nucleotide extensions to further increase the amount of blunt-ended DNA fragments available for adaptor ligation (Costa and Weiner, “Protocols for cloning and analysis of blunt-ended PCR-generated DNA fragments,” PCR Methods Appl 3(5):S95-106, 1994; Costa et al., “Cloning and analysis of PCR-generated DNA fragments,” PCR Methods Appl 3(6):338-45, 1994; Costa and Weiner, “Polishing with T4 or Pfu polymerase increases the efficiency of cloning of PCR products,” Nucleic Acids Res. 22(12):2423, 1994).

Amplification of Nucleic Acid Sequences

Methods for amplifying genetic materials may include whole genome amplification (WGA). (See, for instance, Lovmar et al., “Multiple displacement amplification to create a long-lasting source of DNA for genetic studies,” Hum. Mutat., 27:603-614, 2006). Amplification of nucleic acid sequences may employ any of a number of PCR techniques and non-PCR techniques including, but not limited to, e-PCR, RCA, transcription mediated amplification to target both RNA and DNA for amplification, nucleic acid sequence based amplification (NASBA) for constant temperature amplification, helicase-dependent isothermal amplification, strand displacement amplification (SDA), Q-beta replicase-based methodologies, ligase chain reaction, loop-mediated isothermal amplification (LAMP), and reaction deplacement chimeric (RDC).

DNA Samples

A total of 226 samples were tested for the validation of this new method. These samples, from two groups (described below), were from pedigrees that contained individuals clinically diagnosed with X-linked RP or that showed a pattern consistent with X-linked disease.

De-identified samples for 145 individuals from 52 pedigrees were sourced from the Australian Inherited Retinal Disease Registry and DNA Bank. Samples were sourced from affected and unaffected males and females, including carrier females, from RP families with a clear or suspected X-linked pattern of inheritance.

These DNA samples had previously been Sanger sequenced by the Australian Inherited Retinal Disease Registry (AIRDR); 40 had tested negative for ORF15, while ORF15 mutations had been detected in the remaining 105 samples (54 from affected males and 51 from females with or without symptoms of RP). They were provided for NGS testing, without any accompanying information.

An additional 81 samples from male patients clinically diagnosed with X-linked RP were used for further validation of this method. ORF15 mutations identified in these samples by NGS were later confirmed by targeted Sanger sequencing.

NGS testing of all 226 samples was done by the MVL. Concordance of Sanger sequencing and NGS results for the blind-tested research samples was evaluated by the AIRDR in Australia. The Molecular Vision Laboratory (MVL at Hillsboro, Oreg.) evaluated the clinical samples.

Target Enrichment, NGS Library Preparation, and Sequencing.

Long range PCR (LR-PCR) was used to amplify a 2064 base pair (bp) region of the RPGR gene containing ORF15. DNA (400-500 ng) was amplified in a total reaction volume of 50 using Takara LA Taq DNA polymerase (# RR002M) and forward and reverse primers, AGCAGCCTGAGGCAATAGAA and CAAAATT-TACCAGTGCCTCCT (5′-3′) respectively. The PCR program used was 96° C. for 3 minutes, 30 cycles of 94° C. for 30 seconds, and 68° C. for 15 minutes, followed by 72° C. for 5 minutes, with a final hold at 4° C. LR-PCR products were purified by QIAquick PCR Purification Kit (Qiagen, Hilden, Germany).

NGS libraries were prepared using the Nextera DNA Library Preparation Kit (method 1; Illumina, San Diego, Calif., USA) or the OneTube NGS library preparation kit (Centrillion Technologies, Palo Alto, Calif., USA). The profiles of DNA fragments were analyzed using the DNA 1000 Assay on the Bioanalyzer 2100 (Agilent Technologies, Santa Clara, Calif., USA). Samples were sequenced on Illumina Mi Seq using the 2×150 bp MiSeq Reagent Kit v2 or Illumina HiSeq2500 using TruSeq SBS Kit v3-HS (2×100 bp) plus TruSeq PE Cluster Kit v3-cBot-HS. Samples were allocated with a minimum of 400,000 reads, yielding a target average coverage of at least 20,000 reads for the ORF15 region.

Bioinformatics and Data Analysis

FASTQ files were generated from Illumina's BaseSpace Sequence Hub and aligned using NextGENe by SoftGenetics, LLC (State College, Pa., USA). VCF and BAM files were exported to GeneticistAssistant by SoftGenetics for variant interpretation and mutation identification. Alignment criteria were set to 85% overall base matching percentage and variant detection at 5% minor allele frequency.

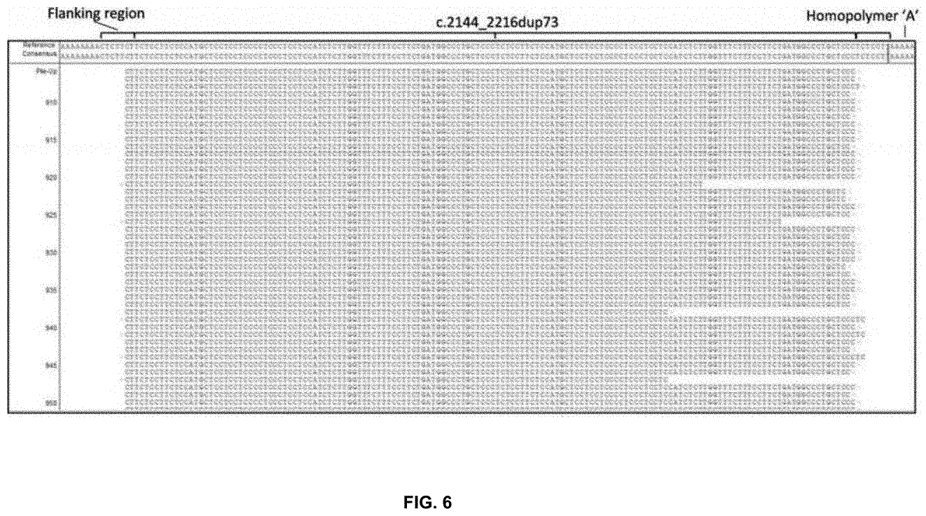

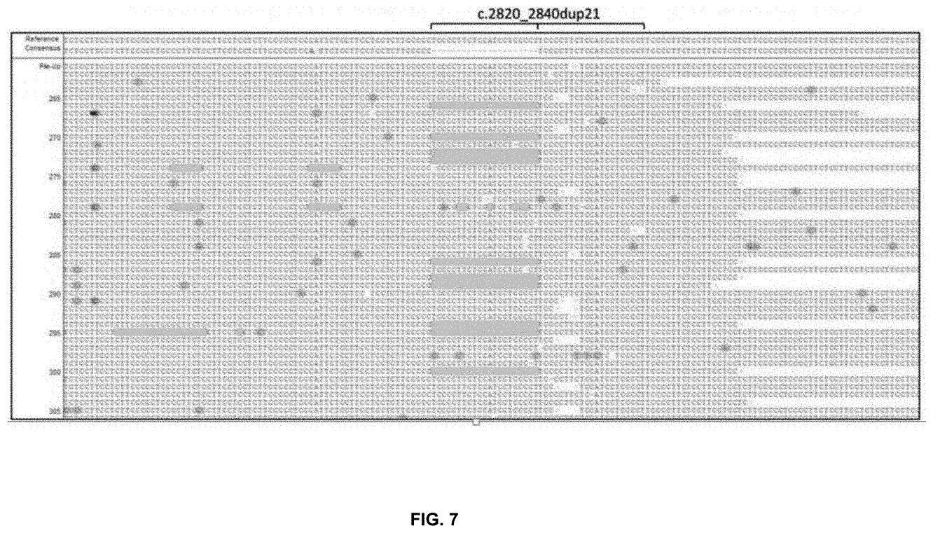

Duplication analysis was done using an artificial reference sequence consisting of 160 bp contigs separated by a 50 bp homopolymer “A.” Contigs were centered on the duplication breakpoint, defined as the junction of the duplicated regions, and provided with a flanking sequence to reach a contig length of 160 bp (see FIG. 6). FIG. 6 shows duplication detection using alignment to an artificial reference sequence. Perfect alignment over this unique duplication junction indicates that presence of c.2144_2216dup within ORF15 of RPGR in this sample. The sequence was generated using a script, stepping through each position from c.2000 to c.3300 and iterating over all duplication sizes from 1 to 200 bp, for a total of 260,000 possible duplications tested. The sequence also can be generated omitting in-frame duplications for frameshift-only analysis. Alignment criteria were set to 100% overall base matching percentage with no allowance for indels. Duplication hits were defined as contigs with >100 aligned reads. Zygosity testing was done on the specific duplication contig only (see FIG. 7), with alignment criteria relaxed to 95% and allowing for indels. FIG. 7 depicts duplication zygosity testing of a mixed sample containing a negative control and a sample homozygous for ORF15 benign duplication, c.2820_2840dup. The wild-type sequence appears as a 21 bp deletion against the reference sequence for the is duplication, while sequence containing c.2920_2840dup, shows complete alignment. FIGS. 6 and 7 are also presented in “Development of High-Throughput Clinical Testing of RPGR ORF15 Using a Large Inherited Retinal Dystrophy Cohort,” J. P. W. Chiang, et al., Invest Ophthalmol Vis Sci. 2018 Sep. 4; 59(11):4434-4440, the disclosures of which are incorporated entirely herein by reference.

NGS Library Preparation from LR-PCR Products

During development of this method for the sequencing of ORF15, the Nextera method was used initially for fragmentation of the LR-PCR product. However, several inconsistencies between Nextera NGS and Sanger sequencing results were detected. These included 12 false-negatives and 1 false-positive. In a further eight cases, mutations were incorrectly identified. Two benign duplication variants also were either incorrectly called or not detected (Table 1). This discordance may be due to the repetitive sequence in ORF15 preventing the transposon-based Nextera fragmentation method from generating a well-represented sequencing library.

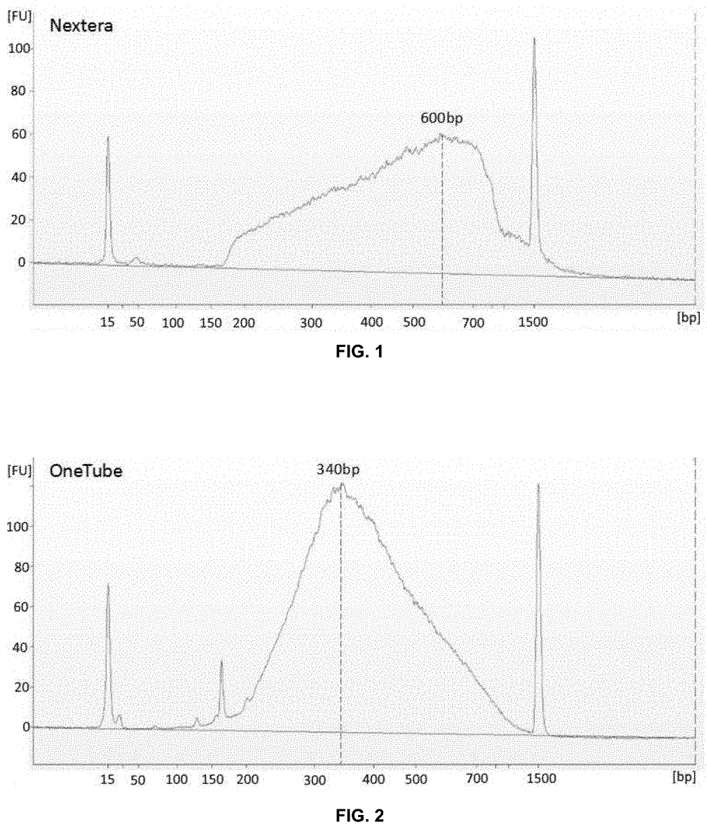

Therefore, a new method—OneTube enzymatic method for library preparation was tested. Distribution of ligated fragment size from Nextera and OneTube fragmentation methods are shown in FIGS. 1 and 2, respectively. The average read length of adapter-ligated fragments was much smaller when using OneTube than that when using Nextera, with peaks observed at 340 and 600 bp, respectively (see FIGS. 1 and 2). DNA fragments were analyzed by BioAnalyzer 2100 DNA 1000 Assay from Agilent. Peaks at 600 and 340 bp are shown for Nextera and OneTube, respectively. Using OneTube NGS, all but two discordant cases from the Nextera method were retested by the OneTube method (see Table 1) randomized with a group of Nextera-Sanger concordant controls. As a result, variants were correctly identified by the OneTube NGS in 21 of the 24 discordant cases. Previous concordant results also were confirmed by the OneTube NGS method.

TABLE 1
Concordance in Variant Data between Sanger Sequencing and NGS of RPGR
ORF15 is Significantly Improved with OneTube-NGS and Duplication Analysis

| | | Reason | | | Next-generation sequencing | | | |
|---|---|---|---|---|---|---|---|---|
| Sample | | for | Sanger sequencing | | Nestera | | One-tube | |
| ID | Gender | testing | Results | Zygosity | result | Zygosity | results | Zygosity |

False negative

| | | | | | | | | |
|---|---|---|---|---|---|---|---|---|
| IRD2809 | F | Obligate | c.2420_2435del16 | HET | Negative | N/A | c.2420_2435del16 | HET |
| | | carrier | | | | | | |
| IRD2551 | F | Possible | c.2420_2435del16 | HET | Negative | N/A | Not tested^{†} | N/A |
| | | carrier | | | | | | |
| IRD2606 | F | Obligate | c.2420_2435del16 | HET | Negative | N/A | c.2420_2435del16 | HET |
| | | carrier | | | | | | |
| IRD4217 | F | Obligate | c.2426_2427delAG | HET | Negative | N/A | c.2426_2427delAG | HET |
| | | carrier | | | | | | |
| IRD4498 | F | Obligate | c.2501delA | HET | Negative | N/A | c.2501delA | HET |
| | | carrier | | | | | | |
| IRD1028 | F | Obligate | c.2635delG | HET | Negative | N/A | c.2635delG | HET |
| | | carrier | | | | | | |
| IRD1035 | F | Obligate | c.2635delG | HET | Negative | N/A | c.2635delG | HET |
| | | carrier | | | | | | |
| IRD1039 | F | Obligate | c.2635delG | HET | Negative | N/A | c.2635delG | HET |
| | | carrier | | | | | | |
| IRD1043 | F | Obligate | c.2635delG | HET | Negative | N/A | c.2635delG | HET |
| | | carrier | | | | | | |
| IRD1076 | F | Obligate | c.2635delG | HET | Negative | N/A | c.2635delG | HET |
| | | carrier | | | | | | |
| IRD1143 | F | Obligate | c.2426_2427delAG | HET | Negative | N/A | c.2426_2427delAG | HET |
| | | carrier | | | | | | |
| IRD2508 | F | Obligate | c.2426_2427delAG | HET | Negative | N/A | c.2426_2427delAG | HET |
| | | carrier | | | | | | |

False positive

| | | | | | | | | |
|---|---|---|---|---|---|---|---|---|
| IRD1275 | F | Possible | Negative | N/A | c.2447delG | HET | Negative | N/A |
| | | carrier | | | | | | |

Mutations called incorrectly

| | | | | | | | | |
|---|---|---|---|---|---|---|---|---|
| IRD2808 | M | Affected | c.2420_2435del16 | HEM | c.2424del | HET | c.2420_2435del16 | HEM |
| IRD2605 | M | Affected | c.2420_2435del16 | HEM | c.2423_2424del | HEM | c.2420_2435del16 | HEM |
| IRD1223 | M | Affected | c.2696_2715del20 | HEM | c.2714_2718del | HEM | c.2696_2715del20 | HEM |
| IRD1282 | M | Affected | c.2696_2715del20 | HEM | c.2714_2718del | HEM | c.2696_2715del20 | HEM |
| IRD1283 | F | Obligate | c.2696_2715del20 | HET | c.2714_2718del | HET | c.2696_2715del20 | HET |
| | | carrier | | | | | | |
| IRD1284 | M | Affected | c.2696_2715del20 | HEM | c.2714_2718del | HET | Not tested^{†} | N/A |
| IRD1305 | F | Possible | c.2696_2715del20 | HET | c.2714_2715del | HET | c.2696_2715del20 | HET |
| | | carrier | | | | | | |
| IRD2885 | F | Obligate | c.2362_2366del5 | HET | c.2358_2362del5 | HET | c.2362_2366del5 | HET |
| | | carrier | | | | | | |
| IRD4036 | M | Affected | c.2144_2216dup73 | HEM | c.2219_2220del | HET | c.2144_2216dup73* | Cannot |
| | | | | | | | | ascertain |
| | | | | | | | | for large |
| | | | | | | | | duplications |

Benign duplications in ORF15

| | | | | | | | | |
|---|---|---|---|---|---|---|---|---|
| IRD1282 | M | Affected | c.2820_2840dup21 | HEM | c.2714_2718del | HET | c.2820_2840dup21* | HEM |
| IRD1275 | F | Possible | c.2447_2661del15 | HET | c.2447delG | HET | c.2447_2661del15 | HET |
| | | carrier | | | | | | |
| IRD4501 | F | Possible | c.2721_2744dup24 | HOM | Negative | N/A | c.2721_2744dup24 | HOM |
| | | carrier | and | | | | and | |
| | | | c.2820_2840dup21 | | | | c.2820_2840dup21* | |

*Duplication analysis.
^{†}DNA sample exhausted.

Coverage of ORF15 and Mutation Detection Accuracy

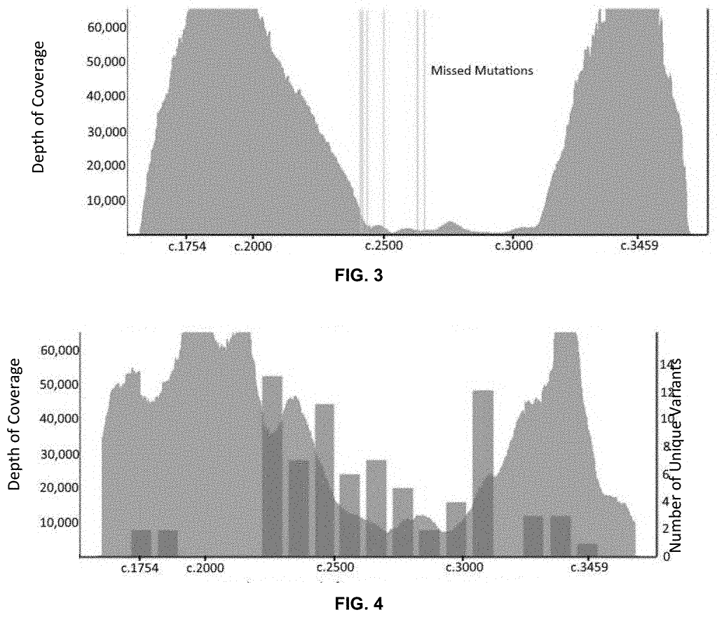

Coverage data from a representative sample can be analyzed and compared. Of the ORF15 mutations identified, 65% were concentrated within the difficult-to-sequence, highly repetitive region (c.2184-3162), for which Nextera and OneTube NGS data highlight a relative lack of coverage (FIGS. 3 and 4). Mutation coverage curves and data for ORF15 of RPGR from NGS of LR-PCR products fragmented with Nextera (FIG. 3) and OneTube (FIG. 4) can be compared by using a representative sample. Vertical lines in FIG. 3 represent the position of missed mutations using Nextera. Rectangle bars in FIG. 4 represent the position and number of unique variants using OneTube (secondary y-axis to the right in FIG. 4).

Minimum coverage when using OneTube NGS (˜6800 reads) was more than 20 times higher than that when using Nextera (˜320 reads), while average coverage of the entire exon was comparable at approximately 36,000 and 32,000 reads for OneTube and Nextera, respectively (Table 2). In setting a coverage threshold of 500 reads as a quality control metric for regions of interest (ROI), OneTube NGS achieved 100% coverage of ORF15, while Nextera NGS achieved 96.8% (Table 2). These results highlight a critical gap in coverage in a region in which ORF15 mutations were concentrated. All Sanger-identified mutations that went undetected using the Nextera method were localized to this region (FIG. 3).

TABLE 2
Comparison of Coverage between Nextera
NGS and One Tube NGS

| | Nextera | OneTube |
|---|---|---|
| Minimum Coverage | 320 | 6,778 |
| Maximum Coverage | 65,535 | 65,535 |
| Average Coverage | 32,048 | 35,752 |
| Percent of ROI with | 96.8% | 100% |
| >500 × coverage | | |
| Number of Bases in | 1,905 | 1,905 |
| ROI | | |

Manual inspection (using NextGENe Viewer) of the mutations initially missed by Nextera-NGS revealed that the mutation sites coincided with highly repetitive areas containing sequence quality issues and alignment difficulties, resulting in many single nucleotide variants being flagged by the software with varying allele frequencies. Poor sequence quality may have masked some of the mutations, highlighting the difficulty in separating true mutations from false-positives under these circumstances. Gaps in coverage also were associated with a higher proportion of sequence data being derived from the ends of reads, where run-specific artifacts commonly are found. When these occur in a significant proportion of available reads at a given location, true-positives can be difficult to distinguish from false-positives. With OneTube-NGS data, we demonstrated that these issues could be overcome with a more uniform distribution of reads staggered across the region of interest, coupled with sufficient depth of coverage to minimize the effect of individual artifacts.

Duplication Analysis

Given the increased prevalence of large duplications within repetitive regions, and the remaining three cases of discordance, duplication analysis was performed using an ORF15-specific in silico array. This method detected the remaining frameshift duplication (c.2144_2216dup, see Table 1) and two benign, in-frame duplications (c.2820_2840dup, c.2721_2744dup, see Table 1), concordant with Sanger sequencing data. Specifically, under strict alignment criteria, approximately 3,000 reads aligned perfectly to the 73 bp (c.2144_2216dup) contig, while less than 10 reads mapped to other contigs (data not shown). Further analysis was successful in determining zygosity for the 21 bp (c.2820_2840dup) and 24 bp (c.2721_2744dup) duplications, but not for the larger 73 bp duplication (c.2144_2216dup). For a 73 bp duplication, the wild-type allele in the case of a heterozygous duplication would be expected to appear as a 73 bp deletion. However, alignment difficulties, owing to deletion size approaching the size of the read length (100 bp), limited the zygosity calling confidence for larger duplications with the present pipeline.

Therefore, the combined method of OneTube fragmentation, supplemented with duplication analysis, may successfully detect all Sanger-identified ORF15 variants among the blind-tested cohort of suspected xlRP pedigrees, in which ORF15 mutations were causative for disease in approximately 50% of cases.

Development of an Accurate ORF15 Clinical NGS Method

The fragmentation method of Nextera NGS method provided insufficient sensitivity and accuracy for sequencing ORF15. Although most of the missed mutations can be detected upon manual inspection, the Nextera NGS method may lack the quality required for robust clinical sequencing. Importantly, this inadequacy was only revealed as a result of studying method disclosed in the present application by testing a large number of Sanger sequenced samples, confirming the importance of clinical validation in NGS method development.

This problem may be solved by using the OneTube method for library preparation, which may achieve 100% specificity and sensitivity with exception of an unclear zygosity calling in one case of a large 73 bp duplication. The marked improvement in accuracy using the OneTube fragmentation method can be attributed to its coverage of this difficult-to-sequence region. The depth of coverage can be a main factor affecting the accuracy of NGS of repetitive regions, such as ORF15. The minimum coverage (˜7000 reads) of the disclosed method is significantly higher than that for recently reported NGS-based ORF15 screening methods (1-2000 reads). Using the disclosed methods of blind-testing against a large number of Sanger-sequenced samples from an xlRP cohort, and comparing the variant detection rate and accuracy of OneTube versus Nextera as shown herein, the amount of coverage required for successful clinical NGS of this region can be determined, and the inadequacy of the Nextera fragmentation method in this instance can be addressed. The disclosed methods may exemplify the importance of such clinical validation in NGS method development.

The OneTube method has been validated against over 50 female samples from suspected xlRP pedigrees. This is important because female samples can be difficult to analyze by Sanger sequencing due to the prevalence of in-frame polymorphic indels. Benefits of being able to successfully analyzing female samples may include informed genetic counseling and the provision of family planning options. For example, the disclosed methods may have noteworthy implications for the analysis of female samples in cases where DNA from an affected male family member may not be available.

Duplication Detection in Highly Repetitive Regions

The short-read length of NGS fragments may also present a challenge in the analysis of highly repetitive regions, in which large deletions and duplications relative to read length may become more common. Large deletions typically can be detected by normal variant calling. However, large duplications can be masked by alignment across the region, with the only distinguishing feature being a single, duplication-specific breakpoint between duplicated regions. Consequently, highly repetitive regions may demand stricter sequencing requirements, and the resulting bottleneck in the bioinformatics pipeline may become increasingly problematic. For example, these repetitive regions may demand stricter sequencing requirements such as higher depth of coverage and lower tolerance for sequencing artifacts.

By utilizing unique, sequence-specific methods that can be adapted to any difficult-to-sequence region in the genome, the disclosed sequencing methods may meet these stringent requirements for high throughput sequencing methods. Out of all the possible sequence variation types in the testing samples, duplications may present a challenge especially as the duplication size becomes large relative to read length. Large duplications may be masked by alignment across the region when the only distinguishing feature is a single duplication-specific breakpoint between the duplicated regions. To isolate alignment to this single duplication-specific breakpoint, an artificial reference sequence can be created consisting of separate contigs corresponding to the regions surrounding specific duplications for all possible duplications in the region (c.2000-3300) of length 1-200 bp for a total of 260,000 possible duplications tested. With this arrangement of artificial contigs and strict alignment criteria, alignment to this reference sequence can serve as a computational array for accurate duplication detection regardless of sequence complexity.

Once the specific duplication is identified, zygosity testing can be done through alignment to the specific duplication breakpoint with standard alignment settings. The wild-type allele in heterozygous cases may appear as a deletion while the allele containing the duplication may align completely. Detection of wild-type alleles may be dependent on the ability to identify deletions within reads, which may depend on the size of the duplication relative to read length. For the duplication cases in the tested cohort and a read length of about 100 bp, zygosity may be correctly identified for a 21 bp (c.2820_2840dup) and a 24 bp duplication (c.2721_2744dup). For a larger, 73 bp, duplication (c.2144_2216dup), the duplication itself may be correctly identified, but zygosity may not be resolved as the reads expected to appear with a deletion may not be aligned using the currently tested pipeline.

The efficacy of the new OneTube sample preparation method may achieve robust coverage of the entirety of ORF15, with about 100% mutation detection sensitivity and specificity for the tested sample population within a standardized clinical pipeline. These results may demonstrate both the weaknesses of previous NGS-based ORF15 sequencing methods, as well as the improvements that the disclosed OneTube method can accomplish. The mutation distribution and coverage data presented in this disclosure can provide a useful benchmark for other NGS-based, clinical testing of hard-to-sequence, repetitive genomic regions, thereby providing comprehensive, accurate, and practical implementation of NGS-based diagnosis for difficult regions within the genome.

Beyond its application to RPGR ORF15, the LR-PCR-based NGS method disclosed herein may show the ability to target any specific region within the genome for accurate, specific, low-cost, and high-coverage sequencing. This method can be applied to finding breakpoints in patients with large deletions identified by array CGH analysis and can form the basis for whole gene sequencing assays for several critical genes in clinical trial pipelines.

Notably, the present methods successfully identified all three Sanger-identified ORF15 duplications that previously were undetected when using the Nextera NGS method. This may distinguish result in detection of large duplications by using high throughput ORF15 screening, which has not been reported or demonstrated previously on clinical samples. This absence in the literature of using NGS methods to detect difficult duplications may be due to the inability of previous NGS methods to detect large duplications.

### Examples

It is understood that the examples and embodiments described herein are for illustrative purposes and that various modifications or changes in light thereof may be suggested to persons skilled in the art and are to be included within the spirit and purview of this application and scope of the claims. Accordingly, the following examples are offered to illustrate, but not to limit, the claimed invention.

For highly repetitive genes, such as, for example, RPGR-ORF15 region (˜2 kb), Mitochondria (˜10 kb) and STRC (˜20 kb), next-generation sequencing can use long-range PCR and OneTube enzymatic fragmentation technology to achieve better, more accurate results. The entire repetitive region can be well-represented with high-quality, random fragmentation to allow for accurate NGS using Illumina HiSeq or MiSeq and subsequent alignment and variant calling.

1. Targeted Amplification of RPGR-ORF15, Mitochondria and STRC

Materials and Equipment

Equipment:

Thermocyclers

Pipettes

Vortex Mixer

1.5 ml centrifuge tube

1.5 ml tubes

96 well Plate or strip tubes

Plate seal

Pipet tips

QX DNA Dilution Buffer

Electrophoresis gel system

0.8 mL 96-well storage plate

NGS Sequencer (Illumina MiSeq)

Materials:

Nuclease-free ultra-pure molecular grade water

QIAquick PCR purification Kit

Takara LA Taq

dNTP Mixture (2.5 mM each)

10×LA PCR Buffer II ((Mg⁺² plus)

LA taq with GC buffer I

Specific forward and reverse primers for RPGR-ORF15, Mitochondria and STRC

End repair Reaction buffer

BSA

Manganese (II) Chloride

Calcium Chloride

End-Prep Enzyme Mix

DNAse I

Blunt TA/Ligase Master Mix

SureSelect Adaptor Oligo Mix

AmpureXP Beads

All-purpose HI-LO DNA Marker/Mass Ladder

DNA 7500 Kit

Tris-HCl

Magnesium Chloride

Certified™ Molecular Biology Agarose

NGS Sequencing Kit (MiSeq v2 Reagent Kit 500 cycles PE)

Fragmentation/End Repair/A-Tailing (FEA) Buffers/Reagents:

5× FEA Buffer (per sample)

| | |
|---|---|
| H₂O | 1.69 μL |
| 10× End Repair Reaction Buffer | 2.0 μL |
| BSA (10 mg/mL) | 0.31 μL |

*10× buffer included with FEA Enzyme 2

FEA Reagent 1

| | |
|---|---|
| H₂O | 99 μL |
| 1M MnCl₂ (final = 10 mM) | 1 μL |

FEA 10× DNase1 In-house Buffer (50 mL)

| | | |
|---|---|---|
| H₂O | 43.50 | mL |
| 1M Tris-HCl, pH 7.5 (final = 100 mM) | 5.0 | mL |
| 1M MgCl₂ (final = 25 mM) | 1.25 | mL |
| 1M CaCl₂ (final = 5 mM) | 250 | μL |

FEA Enzyme 3

| | |
|---|---|
| 10× DNase1 buffer | 499.5 μL |
| 1 U/μL DNase1 (final = 0.001U/μL) | 0.5 μL |

Beads B (per sample)

| | |
|---|---|
| H₂O | 29 μL |
| Beads A | 35 μL |

Long-Range PCR

Takara LA PCR Kit and custom forward and reverse primers for the gene of interest may be needed.
1) Thaw genome DNA (gDNA) sample. Transfer about 500 ng of the sample to strip tube/plate.
2) Thaw 10×LA buffer II (for RPGR-ORF15 and Mitochondria genes), dNTPs, and forward and reverse primers. Keep Taq enzyme on ice. Pulse vortex all reagents and spin down using a pipet to collect reagent at the bottom of the tube.
3) Prepare the following mix for target amplification. Include dead volume for pipetting variance.

| | Volume for 1 | |
|---|---|---|
| Reagents | sample (μl) | |
| Nuclease-free H₂O | 30 | |
| 10× LA buffer II | 5 | |
| dNTPs Mixture (2.5 mM each) | 8 | |
| 10 μM RPGR-ORF15 or | 2 | |
| Mito Forward Primer | | |
| 10 μM RPGR-ORF15 or Mito | 2 | |
| Reverse Primer | | |
| Taq (TaKaRa LA 5 units/μl) | 0.5 | |
| 500 ng gDNA | — | |
| Total Volume | Approx. 50 | μL |

For GC Buffer I (STRC gene), prepare the following mix for target amplification. Include dead volume for pipetting variance:

| Reagents | Volume for 1 sample (μL) | |
|---|---|---|
| Nuclease-free H₂O | 10 | |
| 2× GC Buffer I | 25 | |
| dNTPs Mixture (2.5 mM each) | 8 | |
| 10 μM STRC Forward Primer | 2 | |
| 10 μM STRC Reverse Primer | 2 | |
| Taq (TaKaRa LA 5 units/μL) | 0.5 | |
| 500 ng gDNA | — | |
| Total Volume | Approx. 50 | μL |

4) Gently pulse vortex mix and briefly spin down to collect mix at the bottom of the tube.
5) Aliquot PCR mix to PCR strip tube containing 500 ng of the sample. The final volume may be approximately 50 μl.
6) Gently pulse vortex and centrifuge the tube.
7) Start PCR program on thermal cycler according to the target genes
   I) For PRGR-ORF15 and Mito

| Step | Temperature | Time |
|---|---|---|
| 1 | 96° C. | 3 min |
| 2 | 94° C. | 30 sec |
| 3 | 68° C. | 15 min |

Repeat Step 2 and 3 for a total of 30 cycles

| | | |
|---|---|---|
| 4 | 72° C. | 5 min |
| 5 | 4° C. | Hold |

II) For STRC

| Step | Temperature | Time |
|---|---|---|
| 1 | 94° C. | 2 min |
| 2 | 98° C. | 10 sec |
| 3 | 68° C. | 12 min 10 sec |

Repeat Step 2 and 3 for a total of 36 cycles

| | | |
|---|---|---|
| 4 | 68° C. | 7 min |
| 5 | 4° C. | Hold |

8) After PCR is complete, store at 4° C. for short term storage or at −20° C. for long term storage.

QIAxcel Gel
1) Run the set of samples for RPGR-ORF15 in QIAxcel gel (PCR product size ≤3,000 Kb).
2) For QIAxcel gel use: 3 μL of DNA sample+7 μL of QX DNA Dilution Buffer
3) After running the gel, if the bands in the gel have the size that corresponds to the expected specific primer, go to the next step (column purification). If the gel does not show any band or the band is not in the size that you expect, need to design new primers or do the long-range PCR again (in case the pair of primer is already validated and worked before).
4) Primers for RPGR-ORF15:

Forward:
AGCAGCCTGAGGCAATAGAA
Reverse:
CAAAATTTACCAGTGCCTCCT

Agarose Gel
1) Run the set of samples in agarose gel (PCR product size up to 20,000 Kb).
2) For agarose gel use: 0.4 g of agarose+50 mL of TAE*. Put the flask in the microwave for 1 minute, then wait for about 10 minutes (the flask cannot be too hot) and add 20 μL of DNA Dye. Put the agarose gel+TAE+DNA dye in the gel tank. Wait for about 20 minutes to put the gel tank in the electrophoresis system. Pipete 3 μL of each PCR product to the gel. Run the gel for 45 minutes at 80V. After running the gel, put it on the Digital Gel Image System to take a picture of the gel. Check if all of the samples+primers show a band with the size expected. *To prepare TAE: use 490 mL of water (MiliQ water)+10 mL of TAE (10×).
3) After running the gel, if the bands in the gel have the size that correspond to the expected specific Mito or STRC primers, go to the next step (column purification). If the gel does not show any band or the band is not in the size that you expect, need to design new primers or do the long-range PCR again (in case the pair of primer is already validated and worked before).
4) Primers for Mitochondria:

Mitol (Mt1)-Forward:
AAATCTTACCCCGCCTGTTT
Mitol (Mt1)-Reverse:
AATTAGGCTGTGGGTGGTTG
Mito2 (Mt2)-Forward:
GCCATACTAGTCTTTGCCGC
Mito2 (Mt2)-Reverse:
GGCAGGTCAATTTCACTGGT

5) Primers for STRC:

Forward:
CAGCTCAGAGTTTTTGATAGGGCTTTCA
Reverse:
AGGAAGCAGATCAAAGATTAGTGTCCCTT

Beads Purification

AMPure Beads (Beads A), 200 proof ethanol may be needed. Take out the beads and 70% ethanol from 4° C. Keep them at room temperature at least for 30 mins before use.
1) Add 90 μl AMPure Beads A to each LR-PCR reaction (˜50 μl) and pipet up and down thoroughly to mix the beads and each LR-PCR reaction mixture.
2) Incubate the mixture at room temperature for 5 min to bind DNA to the beads.
3) Place the plate on a magnetic rack and wait until the liquid is clear to capture the beads, usually 5-8 mins. Carefully remove and discard the supernatant.
4) Keep the plate on the magnetic rack and add 200 μl of 70% ethanol to wash the beads.
5) Incubate the plate at room temperature and wait for 30-60 s.
6) Carefully remove and discard the 70% ethanol.
7) Repeat steps 4-6 again. Try to remove the residual ethanol as much as possible without disturbing the beads.
8) Dry the beads at room temperature. To avoid over-drying the beads, drying time should be no longer than 15 mins, usually 7-9 mins.
9) Remove the plate from the magnetic rack.
10) Resuspend the beads in 18 μl biological grade H₂O, pipet up and down for 10-12 times to mix thoroughly.
11) Incubate the plate at room temperature for 5 mins to elute DNA from the beads.
12) Place the plate back on the magnetic rack to capture the beads. Incubate until the liquid is clear, usually 5-8 mins.
13) Transfer 16 μl DNA samples to another plate or 8-stripe tubes for next reaction.

2. Fragmentation/End Repair/A-Tailing (FEA) Reaction

FEA Reaction
1) Thaw reagents 5×FEA Buffer, FEA Reagent 1, and Enzyme 2 on ice. Pulse vortex all reagents and spin down (5 seconds) using a microcentrifuge to collect at the bottom of the tube. Keep ALL reagents on ice.
2) Prepare fresh FEA Enzyme 3 (using DNase and FEA 10× buffer).
3) Prepare FEA master mix on ice in the order listed below:

| Reagents | Amount used for 1 sample |
|---|---|
| 5× FEA Buffer | 4 μL |
| FEA Reagent 1 | 2 μL |
| FEA Enzyme 2 | 1 μL |
| FEA Enzyme 3 | 1.5 μL |
| Total Volume | 8.5 μL |

4) Gently vortex the FEA master mix and spin down briefly (˜5 seconds) to collect mix at the bottom of the tube.
5) On an ice block, transfer 8.5 μL of FEA master mix to a PCR plate.
6) Pipette 11.5 μL of purified gDNA samples. Final volume of FEA reaction may be 20 μL.
7) Gently vortex the tubes and spin down (˜5 seconds) to collect at the bottom.
8) Incubate samples in the pre-programmed thermal cycler program as shown below:

| Step | Temperature | Time |
|---|---|---|
| 1 | 20° C. | 30 min |
| 2 | 65° C. | 30 min |
| 3 | 80° C. | 10 min |
| 4 | 4° C. | Hold |

9) After incubation, microcentrifuge briefly (˜5 seconds) at highest speed to collect at the bottom of the tube and proceed immediately to the Ligation step.

Ligation Reaction
1) Thaw reagent Blunt/TA Ligase Master Mix and SureSelect Adaptor Oligo Mix on ice. Pulse vortex all reagents and spin down (5 seconds) to collect at the bottom of the tube. Keep ALL reagents on ice.
2) Prepare the ligation master mix on ice in the order listed below.

| Reagents | Volume for 1 sample |
|---|---|
| Nuclease-free H₂O | 10 μL |
| Blunt/TA Ligase Master Mix | 10 μL |
| SureSelect Adaptor Oligo Mix | 10 μL |
| Total Volume | 30 μL |

3) Gently vortex the ligation master mix for 5 seconds and briefly centrifuge (˜5 seconds) to collect mix at the bottom of the tube.
4) On an ice block transfer 30 μL of ligation master mix to the tubes containing 20 μL of FEA+gDNA samples.
5) Gently pulse vortex and centrifuge briefly to collect at the bottom of the tube.
6) Incubate samples at room temperature (˜25° C.) for 15 minutes (use a thermal cycle program shown below; and keep hot lid off).

| Step | Temperature | Time |
|---|---|---|
| 1 | 25° C. | 15 min |
| 2 | 4° C. | Hold |

7) After incubation, centrifuge tubes briefly and place on ice immediately. Proceed to next step or store at −20° C.

3. Size Selection

Size Selection Preparation
1. Take out Beads A from 4° C. fridge and leave at room temperature (25° C.) for 15 minutes before proceeding.
2. Prepare 80% ethanol. 400 μL of 70% ethanol will be needed for each sample (2× ethanol washes/sample).
3. Using a multichannel pipette, dilute the DNA library samples with 50 μL H₂O bringing it to a final volume of 100 μL. Pipet up and down several times to mix and centrifuge briefly (5 seconds) to collect sample at the bottom of the tube. If sample ligation product is less than use water to bring final volume to 100 μL.
4. Gently vortex Selection Beads A and Selection Beads B (diluted and previously prepared using Beads A+water) for 10 seconds to fully resuspend the beads. The bead solutions should appear homogenous in color.
5. Aliquot 55 μL of Selection Beads A into a well plate for each sample.
6. Aliquot 64 μL of Selection Beads B into another well plate for each sample.

Size Selection
1. Add 100 μL of the diluted DNA library sample to the corresponding tube containing 55 μL of Beads A. Mix with the pipette to ensure proper homogeneity.
2. Incubate the samples at room temperature for 5 minutes (mixed samples+Beads A).
3. Place samples containing Beads A into a magnetic separation rack and wait for 5 minutes as the beads separate from the supernatant.
4. Carefully transfer 150 μL of the cleared supernatant from each well and add to the corresponding Selection Beads B wells. Avoid disturbing the beads when collecting the supernatant. Immediately mix with the pipette to ensure proper homogeneity.
5. Incubate the plate containing Beads B and supernatant fraction at room temperature for 5 minutes.
6. Place samples containing Beads B into a magnetic separation rack and wait for 5 minutes as the beads B separate from the supernatant.
7. Carefully pipet out and discard the cleared supernatant.
8. Leaving the tubes on the magnetic separation rack, wash the beads with 200 μL of 70% ethanol, wait 30 sec for the beads to settle and then discard the ethanol.
9. Repeat the ethanol wash (Step 8) once more for a total of two washes.
10. Upon completion of the second wash, leave the plate on magnetic separation rack to air dry for 5 minutes at room temperature. The amount of time for air dry may vary. Keep left to air dry until all ethanol has evaporated completely. However, do not over dry the beads as this may affect the yield. Over-dried beads may look dry and cracked. If this occurs, incubate the samples in H₂O (step 11) for an additional 5 minutes and pulse vortex several times during the incubation period.
11. Add 32 μL of nuclease-free H₂O to each well and mix using a pipette for 10 seconds to ensure the beads are fully resuspended in water.
12. Incubate the samples at room temperature for 5 minutes.
13. Place the plate onto the magnetic separation rack and wait for 5 minutes.
14. Carefully transfer 30 μL of the solution into a new plate (this plate has information such as, date, test name, plate #, operator name and CAN) and proceed to the next step. From 30 μL, 15 μL will be used for “Post-sample prep PCR” and the other 15 μL will be saved in the plate “CAN” in case any sample need to be repeated

4. PCR (Post-Sample Prep PCR)

PCR Reaction
1. Thaw the 2× Kapa HiFi HotStart Reaction Mix. Once thawed, keep it on ice.
2. Prepare PCR master mix on ice by adding reagents, 2× Kapa HiFi HotStart Reaction Mix and Nuclease-free water in a 1.5 mL tube.
3. Transfer 15 μL of ligation product to a new strip tube/PCR plate. Store remaining ligation product (15 μL) at −20° C. (Plate named “CAN”).

| Reagents | Volume for 1 sample |
|---|---|
| 2X Kapa HiFi HotStart Reaction Mix | 25 μL |
| Nuclease-free H₂O | 7 μL |
| Total Volume | 32 μL |

4. Gently vortex the PCR master mix for 5 seconds and briefly spin down in a microcentrifuge.
5. On an ice block, aliquot 32 uL of the PCR master mix to the sample+Pipette 3 μL of Index Primer (the same indexes used for Small Panel protocol) for a final volume of 50 μL.
6. Gently vortex and briefly spin down for 5 seconds in a microcentrifuge.
7. Place plate in the thermal cycler and start the ‘post-cap’ PCR reaction program.

Post-PCR Reaction ‘Post-Cap’:

| Step | Temperature | Time |
|---|---|---|
| 1 | 98° C. | 45 sec |
| 2 | 98° C. | 15 sec |
| 3 | 60° C. | 30 sec |
| 4 | 72° C. | 30 sec |

Repeat Step 2 to Step 4 for 10 cycles

| | | |
|---|---|---|
| 5 | 72° C. | 5 min |
| 6 | 10° C. | Hold |

Beads Purification

Repeat the beads purification procedure disclosed above.

Qubit Quantification

Measure the concentrations of each sample with the QUBIT® 2.0 Fluorometer (Life Technology manual) called Post-purification Qubit.

The samples concentrations may be ≥100 ng/mL. If the concentration is lower, the samples may still be run on the MiSeq. However, make note of these samples as these might have a higher chance of failing. If these samples fail on the Miseq run, repeat the entire protocol again for the samples that failed.

Sequencing

Normalize the 2° Post-purification samples to 10 nM and pool them into one tube. After that, diluted part of the 10 nM pool to get a final concentration of 4 nM (for MiSeq run: v2 Reagent Kit 500 cycles PE). Use the diluted samples (4 nM) to run on MiSeq (Check MiSeq run procedure for this final step). The samples from one tube protocol are run together with the samples from the Small Panel protocol.

5. Data Analysis

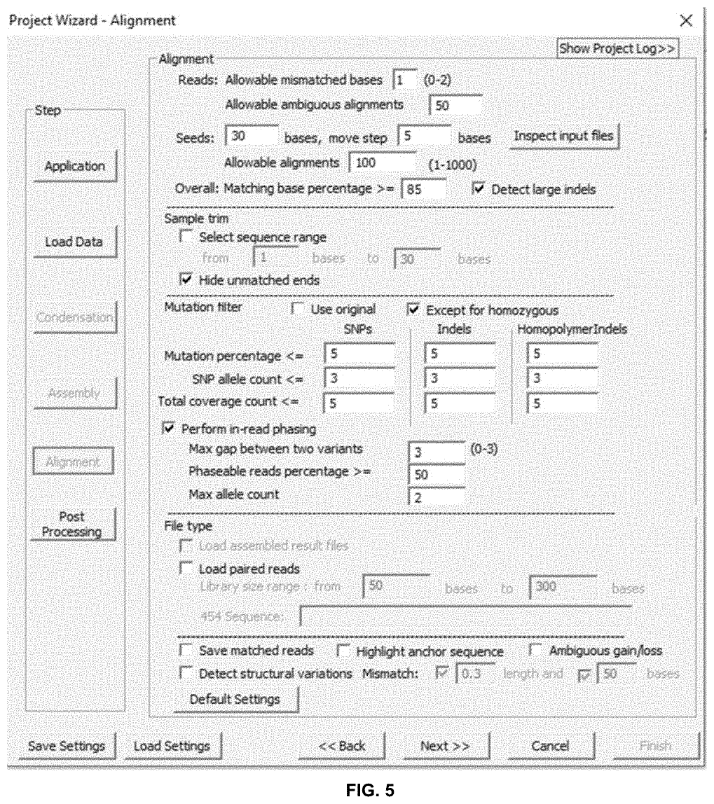

Alignment and variant calling done using NextGENe by Softgenetics. The alignment settings are shown in FIG. 5.

Variants are classified using both public and internal databases according to ACMG guidelines. Primary databases used are ExAC and dbSNP for population information and ClinVar for disease information. For variants of uncertain significance (VOUS), additional references and predictive algorithms may be consulted. Pathogenicity is determined based on ACMG guidelines with frameshift, nonsense, and splice site mutations specifically classified as such. Reported mutations are variants with strong evidence of pathogenicity found in literature or ClinVar. Benign classification is given to variants based on the ACMG criteria (high allele frequency, observation in healthy individual, lack of segregation, etc.) Variants are screened for false positives based on sequence quality and frequency observed.

Mutation confirmation is done using Sanger sequencing or repeating the One tube protocol (if the RPGR-ORF15 region is not covered by Sanger).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of constructing a sequencing library for a region of a target deoxyribonucleic acids (DNA), comprising:
(a) performing a long range polymerase chain reaction (LR-PCR) amplification of the target DNA, thereby producing a plurality of amplified target DNA products; and
(b) fragmenting the plurality of amplified target DNA products by using a deoxyribonuclease (DNase), thereby producing a plurality of fragments of the region of the target DNA;
wherein the region of the target DNA comprises a plurality copies of a repetitive sequence.

2. The method of claim 1, wherein the region of the target DNA further comprises a plurality of variations selected from the group consisted of nucleotide variant, single base substitution, or small indel, transversion, translocation, inversion, deletion, truncation or gene truncation about 1, 2, 3, 4, 5, 6, 7, 8, 9, 10, 15 or 20 nucleotides in length, or a combination thereof.

3. The method of claim 1, wherein the target DNA is RPGR-ORF15 region, mitochondria or STRC.

4. The method of claim 1, wherein the LR-PCR amplification utilizes a plurality of primers, the primers are:
(i) primers for RPGR-ORF15:
Forward:
(SEQ ID NO: 1)
AGCAGCCTGAGGCAATAGAA,
Reverse:
(SEQ ID NO: 2)
CAAAATTTACCAGTGCCTCCT;
or
(ii) primers for Mitochondria:
Mitol (Mt1)-Forward:
(SEQ ID NO: 3)
AAATCTTACCCCGCCTGTTT,
Mitol (Mt1)-Reverse:
(SEQ ID NO: 4)
AATTAGGCTGTGGGTGGTTG,
and/or
Mito2 (Mt2)-Forward:
(SEQ ID NO: 5)
GCCATACTAGTCTTTGCCGC,
Mito2 (Mt2)-Reverse:
(SEQ ID NO: 9)
GGCAGGTCAATTTCACTGGT;
or
(iii) primers for STRC:
Forward:
(SEQ ID NO: 7)
CAGCTCAGAGTTTTTGATAGGGCTTTCA,
Reverse:
(SEQ ID NO: 8)
AGGAAGCAGATCAAAGATTAGTGTCCCTT.

5. The method of claim 1, wherein a minimal depth coverage for the region of the target DNA is more than 900, 1,000, 2,000, 3,000, 4,000, 5,000, or 6,000 reads.

6. The method of claim 5, wherein the minimal depth coverage is about 10, 11, 12, 13, 14, 15, 16, 17, 18, 19, 20, or 21 times higher than another method, the another method using transposase-based Nextera fragmentation in (b).

7. The method of claim 1, wherein the region of the target DNA is more than 1,000, 1,100, 1,200, 1,300, 1,400, 1,500, 1,600, 1,700, 1,800, 1,900, 2,000, 2,100, 2,200, 2,300, 2,400, or 2,500 bp in length.

8. The method of claim 1, wherein the DNase is DNase I.

9. The method of claim 1, further comprising, after (b), end repairing the plurality of fragments of the region of the target DNA, adding a single adenine to the 3′ ends of end repaired fragments using a template independent polymerase; and ligating an adaptor to each end of the repaired fragments comprising a 3′-adenine overhang.

10. A method of detecting at least one mutation within a region of a target deoxyribonucleic acids (DNA), comprising:
(i) constructing the sequencing library for the region of the target DNA according to claim 1;
(ii) sequencing the plurality of fragments of the region of the target DNA in the sequencing library by a next generation sequencing method, thereby acquiring a plurality of reads for the at least one mutation; and
(iii) identifying the at least one mutation.

11. The method of claim 10, wherein the region of the target DNA further comprises a plurality of variations selected from the group consisted of nucleotide variant, single base substitution, or small indel, transversion, translocation, inversion, deletion, truncation or gene truncation about 1, 2, 3, 4, 5, 6, 7, 8, 9, 10, 15 or 20 nucleotides in length, or a combination thereof.

12. The method of claim 10, wherein the target DNA is RPGR-ORF15 region, mitochondria or STRC.

13. The method of claim 10, wherein a minimal depth coverage for the at least one mutation is more than 900, 1,000, 2,000, 3,000, 4,000, 5,000, or 6,000 reads.

14. The method of claim 13, wherein the minimal depth coverage is about 10, 11, 12, 13, 14, 15, 16, 17, 18, 19, 20, or 21 times higher than another method, the another method using transposase-based Nextera fragmentation in (b) when constructing the sequencing library.

15. The method of claim 13, further comprising, after (b) when constructing the sequencing library, end repairing the plurality of fragments of the region of the target DNA, adding a single adenine to the 3′ ends of end repaired fragments using a template independent polymerase; and ligating an adaptor to each end of the repaired fragments comprising a 3′-adenine overhang.

16. The method of claim 10, further comprising, in (iii), conducting duplication analysis.

17. The method of claim 16, wherein the duplication analysis detects a frameshift duplication or an in-frame duplication.

18. The method of claim 16, wherein the duplication analysis comprises using an artificial reference sequence comprising contigs of about 140, 150, 160, 170, or 180 bp in length, wherein each of the contigs centers on a duplication breakpoint, and wherein two adjacent contigs are separated by a homopolymer “A” of about 40, 45, 50, 55, or 60 bp in length.

19. The method of claim 16, wherein the duplication analysis detects a duplication mutation.

20. The method of claim 19, wherein the duplication mutation is not detected by another method, the another method using transposase-based Nextera fragmentation in (b) when constructing the sequencing library.


SEQUENCE LISTING (74 sequences)

SEQ ID NO: 1 - DNA, 20 bp, Artificial Sequence
  Description of Artificial Sequence Synthetic primer
agcagcctgaggcaatagaa

SEQ ID NO: 2 - DNA, 21 bp, Artificial Sequence
  Description of Artificial Sequence Synthetic primer
caaaatttaccagtgcctcct

SEQ ID NO: 3 - DNA, 20 bp, Artificial Sequence
  Description of Artificial Sequence Synthetic primer
aaatcttaccccgcctgttt

SEQ ID NO: 4 - DNA, 20 bp, Artificial Sequence
  Description of Artificial Sequence Synthetic primer
aattaggctgtgggtggttg

SEQ ID NO: 5 - DNA, 20 bp, Artificial Sequence
  Description of Artificial Sequence Synthetic primer
gccatactagtctttgccgc

SEQ ID NO: 6 - DNA, 19 bp, Artificial Sequence
  Description of Artificial Sequence Synthetic primer
gcaggtcaatttcactggt

SEQ ID NO: 7 - DNA, 28 bp, Artificial Sequence
  Description of Artificial Sequence Synthetic primer
cagctcagagtttttgatagggctttca

SEQ ID NO: 8 - DNA, 29 bp, Artificial Sequence
  Description of Artificial Sequence Synthetic primer
aggaagcagatcaaagattagtgtccctt

SEQ ID NO: 9 - DNA, 20 bp, Artificial Sequence
  Description of Artificial Sequence Synthetic primer
ggcaggtcaatttcactggt

SEQ ID NO: 10 - DNA, 173 bp, Artificial Sequence
  Description of Artificial Sequence Synthetic polynucleotide
aaaaaaaactcttcttctccttctccatgctcctcctcccctccctcctccatctcttggtttctttccttctgatggccctgctccctctccttctccatgctcctcctcccctccctcctccatctcttggtttctttccttctgatggccctgctccctctccttaaaaa

SEQ ID NO: 11 - DNA, 148 bp, Artificial Sequence
  Description of Artificial Sequence Synthetic polynucleotide
cttctccttctccatgctcctcctcccctccctcctccatctcttggtttctttccttctgatggccctgctccctctccttctccatgctcctcctcccctccctcctccatctcttggtttctttccttctgatggccctgctccc

SEQ ID NO: 12 - DNA, 149 bp, Artificial Sequence
  Description of Artificial Sequence Synthetic polynucleotide
cttctccttctccatgctcctcctcccctccctcctccatctcttggtttctttccttctgatggccctgctccctctccttctccatgctcctcctcccctccctcctccatctcttggtttctttccttctgatggccctgctccct

SEQ ID NO: 13 - DNA, 147 bp, Artificial Sequence
  Description of Artificial Sequence Synthetic polynucleotide
cttctccttctccatgctcctcctcccctccctcctccatctcttggtttctttccttctgatggccctgctccctctccttctccatgctcctcctcccctccctcctccatctcttggtttctttccttctgatggccctgctcc

SEQ ID NO: 14 - DNA, 117 bp, Artificial Sequence
  Description of Artificial Sequence Synthetic polynucleotide
cttctccttctccatgctcctcctcccctccctcctccatctcttggtttctttccttctgatggccctgctccctctccttctccatgctcctcctcccctccctcctccatctct

SEQ ID NO: 15 - DNA, 146 bp, Artificial Sequence
  Description of Artificial Sequence Synthetic polynucleotide
cttctccttctccatgctcctcctcccctccctcctccatctcttggtttctttccttctgatggccctgctccctctccttctccatgctcctcctcccctccctcctccatctcttggtttctttccttctgatggccctgctc

SEQ ID NO: 16 - DNA, 133 bp, Artificial Sequence
  Description of Artificial Sequence Synthetic polynucleotide
cttctccttctccatgctcctcctcccctccctcctccatctcttggtttctttccttctgatggccctgctccctctccttctccatgctcctcctcccctccctcctccatctcttggtttctttccttct

SEQ ID NO: 17 - DNA, 110 bp, Artificial Sequence
  Description of Artificial Sequence Synthetic polynucleotide
cttctccttctccatgctcctcctcccctccctcctccatctcttggtttctttccttctgatggccctgctccctctccttctccatgctcctcctcccctccctcctc

SEQ ID NO: 18 - DNA, 150 bp, Artificial Sequence
  Description of Artificial Sequence Synthetic polynucleotide
cttctccttctccatgctcctcctcccctccctcctccatctcttggtttctttccttctgatggccctgctccctctccttctccatgctcctcctcccctccctcctccatctcttggtttctttccttctgatggccctgctccctc

SEQ ID NO: 19 - DNA, 167 bp, Artificial Sequence
  Description of Artificial Sequence Synthetic polynucleotide
tctccttcctcttccccctccccttctccttcctcctcttccccctcccattctccttcctcctcttccccctccccttctccatcctccccttccccttctccatcctccccttccccttctccttcctcctcttccccctccccttctccttcctctccttcctc

SEQ ID NO: 20 - DNA, 48 bp, Artificial Sequence
  Description of Artificial Sequence Synthetic oligonucleotide
tctccttcctcttccccctccccttctccttcctcctcttccccctcc

SEQ ID NO: 21 - DNA, 99 bp, Artificial Sequence
  Description of Artificial Sequence Synthetic oligonucleotide
tctccttcctcttccccctccccttctccttcctcctcttccccctcccattctccttcctcctcttccccctccccttctccatcctccccttcccct

SEQ ID NO: 22 - DNA, 66 bp, Artificial Sequence
  Description of Artificial Sequence Synthetic oligonucleotide
tccatcctccccttccccttctccttcctcctcttccccctccccttctccttcctctccttcctc

SEQ ID NO: 23 - DNA, 95 bp, Artificial Sequence
  Description of Artificial Sequence Synthetic oligonucleotide
tctccttcctcttccccctccccttctccttcctcctcttccccctcccattctccttcctcctcttccccctccccttctccatcctccccttc

SEQ ID NO: 24 - DNA, 71 bp, Artificial Sequence
  Description of Artificial Sequence Synthetic oligonucleotide
ccttctccatcctccccttccccttctccttcctcctcttccccctccccttctccttcctctccttcctc

SEQ ID NO: 25 - DNA, 117 bp, Artificial Sequence
  Description of Artificial Sequence Synthetic polynucleotide
tctccttcctcttctccctccccttctccttcctcctcttccccctcccattctccttcctcctcttccccctccccttctccatcctccccttccccttctccatcctccccttcc

SEQ ID NO: 26 - DNA, 111 bp, Artificial Sequence
  Description of Artificial Sequence Synthetic polynucleotide
tctccttcctcttccccctccccttctccttcctcctcttccccctcccattctccttcctcctcttccccctccccttctccatcctccccttccccttctccatcctcc

SEQ ID NO: 27 - DNA, 53 bp, Artificial Sequence
  Description of Artificial Sequence Synthetic oligonucleotide
tccccttctccttcctcctcttccccctccccctctccttcctctccttcctc

SEQ ID NO: 28 - DNA, 96 bp, Artificial Sequence
  Description of Artificial Sequence Synthetic oligonucleotide
tctccttcctcttccccctccccttctccttcctcctcttccccctcccattctccttccccctcttccccctccccttctccatcctccccttcc

SEQ ID NO: 29 - DNA, 68 bp, Artificial Sequence
  Description of Artificial Sequence Synthetic oligonucleotide
tctccatcctccccttccccttctccttcctcctcttccccctccccttctccttcctctccttcctc

SEQ ID NO: 30 - DNA, 108 bp, Artificial Sequence
  Description of Artificial Sequence Synthetic polynucleotide
tctccttcctcttccccctccccttctccttcctcctcttccccctcccattctccttcctcctcttccccctccccttctccatcctccccttccccttctccttcc

SEQ ID NO: 31 - DNA, 53 bp, Artificial Sequence
  Description of Artificial Sequence Synthetic oligonucleotide
cctcttccccctccccttctccttcctcctcttccccctccccttctccttcc

SEQ ID NO: 32 - DNA, 95 bp, Artificial Sequence
  Description of Artificial Sequence Synthetic oligonucleotide
cctcttccccctccccttctccatcctccccttccccttctccatcctccccttccccttctccttcctcctcttccccctccccttctccttcc

SEQ ID NO: 33 - DNA, 102 bp, Artificial Sequence
  Description of Artificial Sequence Synthetic polynucleotide
tctccttcctcttccccctccccttctccttcctcctcttccccctcccattctccttcctcctcttccccctccccttctccatcctccccttccccttct

SEQ ID NO: 34 - DNA, 64 bp, Artificial Sequence
  Description of Artificial Sequence Synthetic oligonucleotide
caccctccccttccccttctccttcctcctcttccccctccccttctccttcctctccttcctc

SEQ ID NO: 35 - DNA, 96 bp, Artificial Sequence
  Description of Artificial Sequence Synthetic oligonucleotide
tctccttcctcttccccctccccttctccttcctcctcttccccctcccattctccttcctcctcttccccctccccttctccatcctccccttcc

SEQ ID NO: 36 - DNA, 110 bp, Artificial Sequence
  Description of Artificial Sequence Synthetic polynucleotide
tctccttcctcttccccctccccttctccttcctcctcttccccctcccattctccttcctccccttccccctccccttctccatcctccccttccccttctccttcctc

SEQ ID NO: 37 - DNA, 88 bp, Artificial Sequence
  Description of Artificial Sequence Synthetic oligonucleotide
tctcctacctcttccccctccccttctccttcctcctcttccccctcccattctccttcctcctcttccccctccccttctccatcct

SEQ ID NO: 38 - DNA, 78 bp, Artificial Sequence
  Description of Artificial Sequence Synthetic oligonucleotide
cccttccccttctccatcctccccttccccttctccttcctcctcttccccctccccttctccttcctctccttcctc

SEQ ID NO: 39 - DNA, 111 bp, Artificial Sequence
  Description of Artificial Sequence Synthetic polynucleotide
tctccttcctcttccccctccccttctccttcctcctcttccccctcccattctccttcctcctcttccccctccccttctccatcctccccttccccttctccttcctcc

SEQ ID NO: 40 - DNA, 53 bp, Artificial Sequence
  Description of Artificial Sequence Synthetic oligonucleotide
cctcttccccctccccttcctcctcttccccctccccttcctcctcttccccc

SEQ ID NO: 41 - DNA, 94 bp, Artificial Sequence
  Description of Artificial Sequence Synthetic oligonucleotide
ccccttctccatcctccccttccccttctccatcctccccttccccttctccttcctcctcttccccctccccttctccttcctctccttcctc

SEQ ID NO: 42 - DNA, 132 bp, Artificial Sequence
  Description of Artificial Sequence Synthetic polynucleotide
tctccttcctcttccccctccccttctccttcctcctcttccccctcccattctccttcctcctcttccccctccccttctccatcctccccttccccttctccatcctccccttccccttctccttcctcc

SEQ ID NO: 43 - DNA, 132 bp, Artificial Sequence
  Description of Artificial Sequence Synthetic polynucleotide
tctccttcctcttccccctcctcttctccttcctcctcttccccctccccttctccttcctcctcttccccctccccttctccatcctccccttccccttctccatcctccccttccccttctccttcctcc

SEQ ID NO: 44 - DNA, 97 bp, Artificial Sequence
  Description of Artificial Sequence Synthetic oligonucleotide
tctccttcctcttccccctccccttctccttcctcctcttccccctcccattctccttcctcctcttccccctccccttctccatcctccccttccc

SEQ ID NO: 45 - DNA, 66 bp, Artificial Sequence
  Description of Artificial Sequence Synthetic oligonucleotide
tccatcctccccttccccttctccttcctcctcttccccctcaccttctccttcctctccttcctc

SEQ ID NO: 46 - DNA, 82 bp, Artificial Sequence
  Description of Artificial Sequence Synthetic oligonucleotide
tctccttcctcttccccctccccttctccttcctcctcttccccctcccattctccttcctcctcttccccctccccttctc

SEQ ID NO: 47 - DNA, 83 bp, Artificial Sequence
  Description of Artificial Sequence Synthetic oligonucleotide
tcctcccctcccccttctccatcctccccttccccctctccttcctcctcttccccctccccttctccttcctctccttcctc

SEQ ID NO: 48 - DNA, 76 bp, Artificial Sequence
  Description of Artificial Sequence Synthetic oligonucleotide
cctcttccccctccccttcctcctcttccccctccccttcctcctcttccccctcaccctcctcctcttcctcttc

SEQ ID NO: 49 - DNA, 65 bp, Artificial Sequence
  Description of Artificial Sequence Synthetic oligonucleotide
ccatcctccccttccccttctccttcctcctcttccccctccccttctccttcctctccttcctc

SEQ ID NO: 50 - DNA, 92 bp, Artificial Sequence
  Description of Artificial Sequence Synthetic oligonucleotide
tctccttcctcttccccctccccttccccttcctcctcttccccctcccattctccctcctcctcttccccctccccttctccatcctcccc

SEQ ID NO: 51 - DNA, 74 bp, Artificial Sequence
  Description of Artificial Sequence Synthetic oligonucleotide
tccccttctccatcctccccttccccttctccttcctcctcttccccctccccttctccttcctctccttcctc

SEQ ID NO: 52 - DNA, 90 bp, Artificial Sequence
  Description of Artificial Sequence Synthetic oligonucleotide
tctccttcctcttccccctccccttctccttcctcctcttccccctcccattctccttcctcctcttccccctccccttctccatcctcc

SEQ ID NO: 53 - DNA, 76 bp, Artificial Sequence
  Description of Artificial Sequence Synthetic oligonucleotide
cttccccttctccatcctccccttccccttctccttcctcctcttccccctccccttctccttcctctccttcctc

SEQ ID NO: 54 - DNA, 90 bp, Artificial Sequence
  Description of Artificial Sequence Synthetic oligonucleotide
tctccttcctcttccccctccccttcgccttcctcctcttccccctcccattctccttcctcctcttccccctccccttctccatcctcc

SEQ ID NO: 55 - DNA, 76 bp, Artificial Sequence
  Description of Artificial Sequence Synthetic oligonucleotide
cttccccttctccatcctccacttccccttctccttcctcctcgaccccctccccttctccttcctctcctccctc

SEQ ID NO: 56 - DNA, 99 bp, Artificial Sequence
  Description of Artificial Sequence Synthetic oligonucleotide
tctccttcctcttccccctccccttctccttcctcctcttccccctcccattctcctccctcctcttccccctccccttctccatcctccccttcccct

SEQ ID NO: 57 - DNA, 111 bp, Artificial Sequence
  Description of Artificial Sequence Synthetic polynucleotide
tctccttcctcttccccctccccttctccttcctcctcttccccctccccttctccttcctcctcttccccctccccttctccatcctccccttccccttctccttcctcc

SEQ ID NO: 58 - DNA, 90 bp, Artificial Sequence
  Description of Artificial Sequence Synthetic oligonucleotide
tccccttcctcttccccctccccttctccttcctcctcttccccctcccattctccttcctcctcttccccctccccttctccatcctcc

SEQ ID NO: 59 - DNA, 76 bp, Artificial Sequence
  Description of Artificial Sequence Synthetic oligonucleotide
cttccccttctccatccttcccttccccttctccttcctcctcttccccctccccttctccttcctctccttcctc

SEQ ID NO: 60 - DNA, 106 bp, Artificial Sequence
  Description of Artificial Sequence Synthetic polynucleotide
tccccttcctcttccccctccccctctccttcctcctcttccccctcccattctccttcctcctcttccccctccccttctccatcctccccttccccttctcctt

SEQ ID NO: 61 - DNA, 166 bp, Artificial Sequence
  Description of Artificial Sequence Synthetic polynucleotide
tctccttcctcttccccctccccttctccttcctcctcttccccctaccattctccttctcctcttccccctccccttctccatcctccccttccccttctccatcctccccttccccttctccttcctcctcttccccctccccttctccttcccctccttcctc

SEQ ID NO: 62 - DNA, 90 bp, Artificial Sequence
  Description of Artificial Sequence Synthetic oligonucleotide
cctcttccccctccccttctccttcctcctcttccccctcccattctccttcctcctcttccccctccccttctccatcctccccttccc

SEQ ID NO: 63 - DNA, 66 bp, Artificial Sequence
  Description of Artificial Sequence Synthetic oligonucleotide
tccatcctccccttccccttctccttcctcctcttccccctccccttctccttcctccccttcctc

SEQ ID NO: 64 - DNA, 127 bp, Artificial Sequence
  Description of Artificial Sequence Synthetic polynucleotide
tctccttcctcttcctctcctccccctcccattctccttcctcctcttccccctccccttctccatcctccccttccccttctccttcctcctcttccccctccccttctccttcctctccttcctc

SEQ ID NO: 65 - DNA, 93 bp, Artificial Sequence
  Description of Artificial Sequence Synthetic oligonucleotide
tctccttcctcttccccctccccttctccttcctcctcttccccctcccattctccttcctcctcttccccctccccttctccatcctcccct

SEQ ID NO: 66 - DNA, 73 bp, Artificial Sequence
  Description of Artificial Sequence Synthetic oligonucleotide
ccccttctccatcctccccttccccttctccttccacctcttccccctccccttctccttcctctccttcctc

SEQ ID NO: 67 - DNA, 166 bp, Artificial Sequence
  Description of Artificial Sequence Synthetic polynucleotide
tctccttcctcttccccctccccttctccttcctcctcttccccctcccattctccttcctcctcttccccccccccctctccatcctccccctcccctttttcctctccccttccccttctccttcctcctcttccccctccccttctccttcctctccttcctc

SEQ ID NO: 68 - DNA, 91 bp, Artificial Sequence
  Description of Artificial Sequence Synthetic oligonucleotide
tctccttcctcttccccctccccttctccttcctcctcttccccctcccattctccttcctcctcttccccctccccttctccatcctccc

SEQ ID NO: 69 - DNA, 75 bp, Artificial Sequence
  Description of Artificial Sequence Synthetic oligonucleotide
ttccccttctccatcctccccttccccttctccttcctcctcttccccctccccttctccttcctctccttcctc

SEQ ID NO: 70 - DNA, 103 bp, Artificial Sequence
  Description of Artificial Sequence Synthetic polynucleotide
tctccttcctcttccccctccccttctccttcctcctcttccccctcccattctccttcctcctcttccccctccccttctccatcctccccttccccttctc

SEQ ID NO: 71 - DNA, 63 bp, Artificial Sequence
  Description of Artificial Sequence Synthetic oligonucleotide
atcctccccttccccttctccttcctcctcttccccctccccttctccttcctctccttcctc

SEQ ID NO: 72 - DNA, 134 bp, Artificial Sequence
  Description of Artificial Sequence Synthetic polynucleotide
tctccttcctcttccccctccccttctccttcctcctcttccccctcccattctccttcctcctcttccccctccccttctccatcctccccttccccttctccatcctccccttccccttctccttcctcctc

SEQ ID NO: 73 - DNA, 96 bp, Artificial Sequence
  Description of Artificial Sequence Synthetic oligonucleotide
cccccttcctcttccccctccccttccccttcctcctcttccccctcccattctccttcctcctcttccccctccccttctccatcctccccttcc

SEQ ID NO: 74 - DNA, 95 bp, Artificial Sequence
  Description of Artificial Sequence Synthetic oligonucleotide
tctccttcctcctcttccccctccccttctccatcctccccttccccttctccttcctcctcttccccctccccttctccttcctctccttcctc